(19) United States
(12) Patent Application Publication
(10) Pub. No.: US 2026/0104402 A1
(43) Pub. Date: Apr. 16, 2026

(54) SYSTEM AND METHOD FOR DECODING ELEMENTAL COMPOSITIONS OF MULTI-COMPONENT ALLOYS USING DENSITY FINGERPRINTING AND AN AUTOPOIETIC E-PHANTOM DATABASE

(71) Applicants: RATHORE PRATIBHA, Moradabad (IN); RATHORE BHARATI, Moradabad (IN); RATHORE BHUVAN CHANDRA, Moradabad (IN); RATHORE JAI HIND, Moradabad (IN)

(72) Inventors: RATHORE PRATIBHA, Moradabad (IN); RATHORE BHARATI, Moradabad (IN); RATHORE BHUVAN CHANDRA, Moradabad (IN); RATHORE JAI HIND, Moradabad (IN)

(21) Appl. No.: 19/229,325

(22) PCT Filed: Jun. 1, 2024
(86) PCT No.: PCT/IB2024/055369

§ 371 Date: Jun. 5, 2025

(30) Foreign Application Priority Data
Jun. 2, 2023 (IN) 202311038187

(51) Int. Cl. G01N 33/2028; C22C 1/00; C22C 33/00

(52) U.S. Cl. CPC G01N 33/2028; C22C 1/00; C22C 33/00

(57) ABSTRACT

The present invention discloses a Density Decoding System (DDS) (100) and associated methods for determining elemental percent compositions of multi-component alloys from their densities. The DDS (100) synergistically combines modified Archimedes density equations, computation of Probable Iso-density Compositions (PICs), identification of Concordant Compositions (CCs), mapping to an autopoietic e-Phantom database (170) containing Quantized Field of Compositions (QFCs) in a Vast Alloy Space (VAS), and iterative refinement (195) to determine the True Composition (TC) of unknown alloy (105). The system (100) comprises an input interface (130, 130a), a processing unit with mathematical (175), compiler (180), and analyzer (190) modules, and an output interface (230, 130a). The method involves receiving input density (120) and calibration parameters, computing PICs, compiling PIC series, identifying CCs, mapping to the e-Phantom database, iteratively refining (195) to determine the TC, and mining the database for new alloys.

### TECHNICAL FIELD

The present invention relates generally to the field of materials science and engineering, particularly to the analysis, characterization, design and discovery of multi-component alloys. More specifically, it relates to a system and method for determining the elemental percent compositions of multi-component alloys from their measured densities using a novel density fingerprinting approach. The invention further extends to the design and discovery of new alloys with desired properties using an autopoietic quantized e-Phantom database containing Quantised Field of Compositions (QFCs) in Vast Alloy Space (VAS).

### BACKGROUND

Determining the chemical compositions of alloys has been a longstanding challenge, especially for non-binary alloys containing three or more constituent elements. The classical Archimedes density method, while effective for binary alloys, becomes mathematically unsolvable for non-binary alloys due to the underdetermined system of equations that arises.

Furthermore, the densities of non-binary alloys are always associated with an infinite number of Probable Iso-density Compositions (PICs). Discretizing this infinite set into a countable finite set poses a computationally NP-hard problem. Even if the PICs could be enumerated, conclusively determining the True Composition (TC) from the list of PICs is incredibly challenging and unresolved to date.

Modern analytical techniques like spectroscopy and radiation methods enable compositional analysis of multi-component alloys. However, these are often destructive, limited to surface analysis, expensive, and non-ergonomic. In contrast, the simplicity, cost-effectiveness, non-destructive nature and bulk analysis capability of the Archimedes density method remains appealing if its limitations for non-binary alloys can be addressed. Unfortunately, determining the elemental compositions of multi-component alloys using the Archimedes density method has remained an unresolved challenge since its inception in 240 BC.

The present disclosure describes a Density Decoding System (DDS) (100) and associated methods that resolve these challenges and enable accurate determination of elemental percent compositions of multi-component alloys directly from their densities. The DDS (100) introduces a novel density fingerprinting approach that synergistically combines modified Archimedes density equations, PICs computation, Concordant Composition (CC) identification, an autopoietic e-Phantom database mapping, and an iterative refinement process to conclusively ascertain the actual alloy composition. Furthermore, the DDS (100) leverages the autopoietic quantized e-Phantom database (170) for the analysis, characterization, design, and discovery of new alloys with desired properties.

### SUMMARY

The primary objective of the present invention is to provide a system and method for accurately determining the elemental percent compositions of multi-component alloys directly from their measured densities. This is achieved through a novel density fingerprinting approach implemented in the Density Decoding System (DDS) (100). A secondary objective is to leverage the DDS (100) and its autopoietic quantized e-Phantom database (170) for the design and discovery of new alloys with desired properties.

In one aspect, a Density Decoding System (DDS) (100) for determining elemental percent compositions of alloys from their densities is disclosed. The system (100) comprises:
(a) an input interface (130, 130a) for receiving an input density (120) of an unknown alloy (105) from a densitometer (110) or other means, and calibration parameters including the Metals program (140), constituent elements along with their standard densities (150), and an iterative step (default i=1) (160);
(b) a computing unit (165) operatively coupled with the input interface (130, 130a) and configured to execute:
   a mathematical module (Maths-mill) (175) for computing Probable Iso-density Compositions (PICs) for the input density (120) of the unknown alloy (105) using modified Archimedes density equations, wherein the PICs are computed in a plurality of series based on combinatorial subset ⁿC₂ of the constituent elements (150), where n is the number of constituent elements (150), and wherein each PIC series is computed by iteratively feeding quantized Successively increasing Predefined Imaginary Numerical values (SPIN-values) for percent mass fractions of the 3rd, 4th or additional constituent elements into the modified Archimedes density equations;
   a compiler module (180) for compiling the computed PICs into respective PIC series to constitute a real-time database (i-Database) (185) and mapping the PIC series in the Quantized Field of Compositions (QFCs) in an autopoietic e-Phantom database (170) to visualize them as Isopycnic Regions (IRs) in an Alloy Space (AS) of a Vast Alloy Space (VAS), wherein the e-Phantom database (170) is generated instantly upon selecting the constituent elements (150) and iterative step (160), and comprises composition pockets each having an associated effective density that encodes a True Composition (TC) of the alloy constituting a Density Genome (DG) for the pocket;
   an analyzer module (190) for identifying Concordant Compositions (CCs) among the plurality of PIC series in the Isopycnic Regions (IRs) as the Most Probable Composition (MPC) of the unknown alloy (105), mapping density of the MPC to the effective densities in the QFCs pockets of the autopoietic e-Phantom database (170), and iteratively refining (195) the mapped composition until convergence to determine the True Composition (TC) of the unknown alloy (105); and for mining the e-Phantom database (170) to identify unexplored composition pockets as potential new alloys;
(c) an output interface (230, 130a) operatively coupled with the computing unit (165) for displaying (225) the determined True Composition (TC) as characterisation (210) of the unknown alloy (105), alloy designing data (200), and one or more visualizations (220) of the PICs, CCs, density fingerprints, quantized e-Phantom database (170) mappings, alloy composition maps highlighting unexplored pockets, spectral fingerprints, wave interference patterns, 1D/2D/3D projections, and combinations thereof;
   wherein, the system (100) is configured to execute the input interface (130, 130a), the mathematical module (Maths-mill) (175), compiler module (180), analyzer module (190), and output interface (230, 130a) without prior knowledge of the constituent elements (150) of the unknown alloy (105) for the n-elements program (140).

In another aspect, a method for determining elemental percent compositions of multi-component alloys from their densities is disclosed. The method comprises:
(a) receiving an input density (120) of an unknown alloy (105) from a densitometer (110) or any other source, and calibration parameters including a Metals program (140) specifying number of constituent elements (150), the constituent elements (150) along with their standard densities, and an iterative step (default i=1) (160);
(b) computing Probable Iso-density Compositions (PICs) of the alloy for the input density (120) using modified Archimedes density equations, wherein the PICS are computed in a plurality of series based on combinatorial subset ⁿC₂ of the constituent elements (150), where n is the number of constituent elements (150), and wherein each PIC series is computed by iteratively feeding quantized Successively increasing Predefined Imaginary Numerical values (SPIN-values) for percent mass fractions of the 3rd, 4th or additional constituent elements into the modified Archimedes density equations;
(c) compiling the computed PICs into respective PIC series to constitute a real-time database (i-Database) (185) and mapping the PIC series in the Quantized Field of Compositions (QFCs) in an autopoietic e-Phantom database (170) to visualize them as Isopycnic Regions (IRs) in an Alloy Space (AS) of a Vast Alloy Space (VAS), wherein the e-Phantom database (170) is generated instantly upon selecting the constituent elements (150) and iterative step (160), and comprises composition pockets each having an associated effective density that encodes a True Composition (TC) of the alloy constituting a Density Genome (DG) for the pocket;
(d) identifying Concordant Compositions (CCs) among the plurality of PIC series in the Isopycnic Regions (IRs), wherein the CCs replicate across the PIC series and represent a Most Probable Composition (MPC) of the alloy;
(e) mapping the MPC to an effective density in the QFCs pockets of the autopoietic e-Phantom database (170), wherein the effective density encodes a True Composition (TC) of the alloy for its corresponding composition pocket in the database;
(f) iteratively refining (195) the MPC by mapping its density to the effective density at successively higher resolution levels of the e-Phantom database (170) until convergence to determine the True Composition (TC) of the alloy;
(g) mining the e-Phantom database (170) to identify unexplored composition pockets representing potential new alloys; and
(h) outputting the determined True Composition (TC) as characterisation (210) of the unknown alloy (105), alloy designing data (200), and one or more visualizations (220) of the PICs, CCs, density fingerprints, quantized e-Phantom database (170) mappings, alloy composition maps highlighting unexplored pockets, spectral fingerprints, wave interference patterns, 1D/2D/3D projections, and combinations thereof;
   wherein, steps (a) through (h) are performed without prior knowledge of the constituent elements (150) of the unknown alloy (105) for the n-elements program (150).

In yet another aspect, a non-transitory computer readable medium with computer executable instructions stored thereon for determining elemental percent compositions of multi-component alloys from their densities is disclosed, wherein the instructions, when executed by a processor (134, 134a), cause the processor to perform the method.

In yet another aspect, a computer program product comprising a non-transitory computer readable medium having a computer readable program embodied therein is disclosed. The computer readable program, when executed on a computing device, causes the computing device to execute the method for determining elemental percent compositions of unknown alloys (105) from their densities.

In another aspect, a computer-implemented method for determining elemental percent compositions of multi-component alloys from their densities using the Density Decoding System (DDS) (100) is disclosed. The computer-implemented method comprises:
(a) receiving, via an input interface (130, 130a), an input density (120) of unknown alloy (105) and calibration parameters including a Metals program (140) specifying number of constituent elements, the constituent elements (150) along with their standard densities, and an iterative step (160);
(b) computing, using a processor (134, 134a) executing a mathematical module (Maths-mill) (175), PICs of the unknown alloy (105) for the input density (120) using modified Archimedes density equations, wherein the PICs are computed in a plurality of series based on combinatorial subset ⁿC₂ of the constituent elements (150), where n is the number of constituent elements (150), and wherein each PIC series is computed by iteratively feeding quantized Successively increasing Predefined Imaginary Numerical values (SPIN-values) for percent mass fractions of additional constituent elements (150) into the modified Archimedes density equations;
(c) compiling, using the processor executing a compiler module (180), the computed PICs into respective PIC series to constitute a real-time database (185) and mapping the PIC series in the QFCs in an autopoietic e-Phantom database (170) to visualize them as Isopycnic Regions (IRs) in an Alloy Space (AS) of a Vast Alloy Space (VAS);
(d) identifying, using the processor executing an analyzer module (190), CCs among the plurality of PIC series, mapping a density of the CCs to the effective densities in the QFCs pockets of the autopoietic e-Phantom database (170), and iteratively refining (195) the mapped composition until convergence to determine the True Composition (TC) of unknown alloy (105);
(e) mining, using the processor executing the analyzer module (190), the e-Phantom database (170) to identify unexplored composition pockets as potential new alloys; and
(f) outputting, via an output interface (230, 130a), the determined True Composition (TC) of the unknown alloy (105) as characterisation (210), alloy designing data (200), and one or more visualizations (220).

In a further aspect, a computer system for determining elemental percent compositions of multi-component alloys from their densities using the System/Density Decoding System (DDS) (100) is disclosed. The system comprises:
(a) a processor (134, 134a);
(b) an input interface (130, 130a) coupled to the processor (134, 134a) and configured to receive an input density (120) of unknown alloy (105) and calibration parameters including a Metals program (140) specifying number of constituent elements, the constituent elements (150) along with their standard densities, and an iterative step (160);
(c) an output interface (230, 130a) coupled to the processor (134, 134a); and (d) a memory (133, 133a) coupled to the processor (134, 134a), wherein the memory (133, 133a) stores instructions which, when executed by the processor (134, 134a), cause the processor to:
   compute Probable Iso-density Compositions (PICs) of the alloy (105) for the input density (120) using modified Archimedes density equations, wherein the PICs are computed in a plurality of series based on combinatorial subset ⁿC₂ of the constituent elements (150), where n is the number of constituent elements (150), and wherein each PIC series is computed by iteratively feeding quantized Successively increasing Predefined Imaginary Numerical values (SPIN-values) for percent mass fractions of additional constituent elements (150) into the modified Archimedes density equations;
   compile the computed PICs into respective PIC series to constitute a real-time database (185) and map the PIC series in the Quantized Field of Compositions (QFCs) in an autopoietic e-Phantom database (170) to visualize them as Isopycnic Regions (IRs) in an Alloy Space (AS) of Vast Alloy Space (VAS);
   identify Concordant Compositions (CCs) among the plurality of PIC series, map a density of the CCs to the effective densities in the QFCs pockets of the autopoietic e-Phantom database (170), and iteratively refine (195) the mapped composition until convergence to determine the True Composition (TC) of the alloy (105);
   mine the e-Phantom database (170) to identify unexplored composition pockets as potential new alloys; and
   output, via the output interface (230, 130a), the determined True Composition (TC) of unknown alloy (105) as characterisation (210), alloy designing data (200), and one or more visualizations (220).

The density fingerprinting approach leverages several key insights:
(a) Density is a fundamental property that encodes compositional information of alloys;
(b) Modifying the Archimedes density equations allows incorporation of additional constituent elements;
(c) The infinite number of PICs associated with densities of multi-component alloys can be successfully contained by putting them on a percent scale;
(d) Quantized PICs can be computed by iteratively inputting SPIN-values;
(e) An autopoietic e-Phantom database (170) containing QFCs can be generated for any given set of constituent elements;
(f) CCs among PIC series represent the MPC of the alloy;
(g) Iterative refinement (195) of the MPC by mapping its density to effective densities in the QFCs leads to the conclusive determination of the TC;
(h) Conclusive certainty can be achieved from the probabilities;
(i) The quantized e-Phantom database (170) can be mined for unexplored compositions representing new alloys with potentially novel properties.

### BRIEF DESCRIPTION OF THE DRAWINGS

FIG. 1 illustrates the block diagram of the Density Decoding System (DDS) (100) according to an embodiment of the present invention.

FIG. 2 illustrates the stand-alone (135) computing setup for the DDS (100) according to an embodiment.

FIG. 3 illustrates the remote/server/cloud (137) computing setup for the DDS (100) according to an embodiment.

FIG. 4 illustrates the algorithm flow in the computing unit (165) of DDS (100) according to an embodiment.

FIG. 5 illustrates the algorithm flow in client machine (136) interface unit (130a) of a remote/server/cloud (137) computing setup for the DDS (100) according to an embodiment.

FIG. 6 illustrates the algorithm flow in client machine (136) processing unit (134) for a remote/server/cloud (137) computing setup for the DDS (100) according to an embodiment.

FIG. 7 illustrates the algorithm flow in a stand-alone (135) computing setup for the DDS (100) according to an embodiment.

FIGS. 8A-J illustrate the DDS (100) interfaces showcasing various results and visualizations for a ternary alloy example.

FIGS. 9A-I illustrate the DDS (100) interfaces showcasing various results and visualizations for a quaternary alloy example.

FIGS. 10A-F illustrate the DDS (100) interfaces showcasing various results and visualizations for a senary alloy example.

FIG. 11 exhibits the complete spectrum and fingerprints of pure elements, binary alloys, and ternary alloys obtained from 3, 4, 5 & 6-Metals Programs.

FIG. 12 exhibits the complete spectrum and fingerprints of quaternary, quinary and senary alloys obtained from various Metals Programs.

FIGS. 13A-B illustrate the DDS (100) interfaces showcasing the composition spectrum and fingerprints for an octonary alloy example.

FIGS. 14A-C illustrate the DDS (100) interfaces showcasing the composition spectrum and fingerprints for a ternary alloy example.

FIGS. 15A-C illustrate the DDS (100) interfaces showcasing the composition spectrum and fingerprints for a quaternary alloy example.

### DETAILED DESCRIPTION

The present invention relates to a Density Decoding System (DDS) (100) and associated methods for determining the elemental percent compositions of multi-component unknown alloys (105) directly from their measured densities (120). The DDS (100) leverages a novel density fingerprinting approach that synergistically combines:
Modified Archimedes density equations to include additional constituent elements (150)
Computation of quantized Probable Iso-density Compositions (PICs)
Compilation of PICs series to constitute a real-time database (i-Database) (185) and mapping it as Isopycnic Regions (IRs) in Alloy Space (AS) of Vast Alloy Space (VAS) in the autopoietic e-Phantom database (170) containing Quantised Field of Compositions (QFCs)
Identification of Concordant Compositions (CCs) among PIC series as the Most Probable Composition (MPC)
Mapping the MPC to an autopoietic e-Phantom database (170) containing Quantised Field of Compositions (QFCs) in its Vast Alloy Space (VAS).
Iterative refinement (195) of the MPC to determine the True Composition (TC) of unknown alloy (105)
Mining of the quantized e-Phantom database (170) for new alloys with desired properties

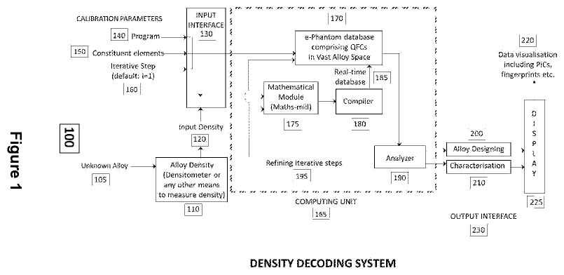

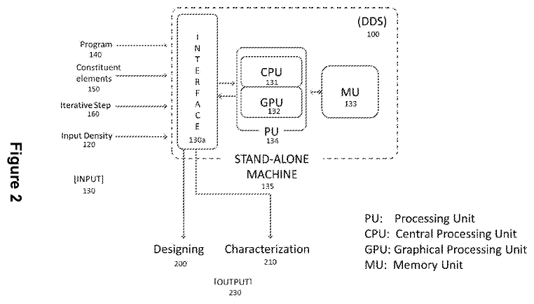

FIG. 1 illustrates a block diagram of the DDS (100) according to an embodiment. The system comprises:
An input interface (130, 130a) for receiving the alloy density (120) of unknown alloy (105) from a densitometer (110) or other means, along with calibration parameters including the Metals program (140), constituent elements (150) along with their standard densities, and the iterative step (default i=1) (160).
A DDS computing unit (165) consisting of:
   A mathematical module (Maths-mill) (175) that computes the PICs using modified Archimedes density equations for the input density (120) of the unknown alloy (105) in multiple PICs series based on the ⁿC₂ combinations of constituent elements (150). The PICs are computed iteratively using SPIN-values supplied for each additional constituent element.
   A compiler module (180) that organizes the computed PICs into respective PIC series to constitute a real-time database (i-Database) (185) for the input density (120), and maps the PIC series in the Quantized Field of Compositions (QFCs) to visualize them as Isopycnic Regions (IRs) in the Alloy Space (AS) of the Vast Alloy Space (VAS) in the autopoietic e-Phantom database (170).
   An analyzer module (190) that identifies Concordant Compositions (CCs) among the PIC series as the Most Probable Composition (MPC). It maps the MPC to effective densities in the QFCs pockets and iteratively refines (195) the composition until convergence to determine the True Composition (TC) of the unknown alloy (105).
An output interface (230, 130a) that displays (225) the decoded True Composition (TC) of unknown alloy (105) as characterization data (210) and alloy designing data (200), along with visualizations (220) of the PICs, CCs, alloy fingerprints, and other data.

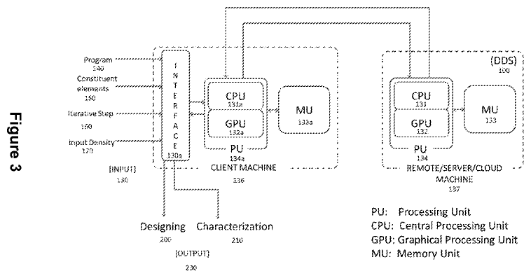

The DDS (100) can be implemented either on a stand-alone device (135) (FIG. 2) or through a client machine (136) and remote/server/cloud (137) setup (FIG. 3). In both configurations, the computing unit leverages CPU (131, 131a) and GPU (132, 132a) processors along with memory units (133, 133a) to optimally execute the math-intensive PIC computations and database mapping operations.

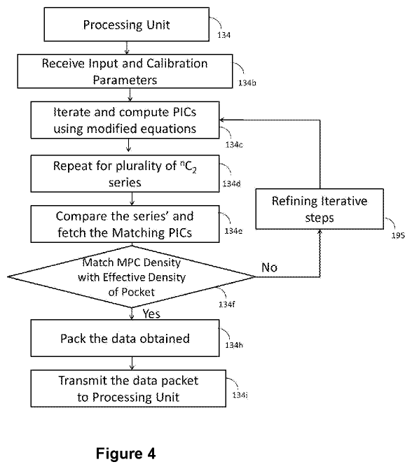

FIG. 4 depicts the algorithm flow in the computing unit (165) of DDS (100). Upon receiving the input density, and calibration parameters (134b), the system iterates to compute PICs using the modified Archimedes density equations (134c). It repeats this for a plurality of the ⁿC₂ PIC series (134d). The series are then compared to identify the Concordant Compositions (CCs) (134e). The processing unit (134) maps the CC density to effective densities in the QFCs pockets of the e-Phantom database (170) and determines if it matches (134f). If yes, the CC is confirmed as the True Composition (TC) of the unknown alloy (105), the data is packed (134h) and transmitted to the processing unit (134i). If not, the iterative step (160) is refined (195) and the process repeats until convergence.

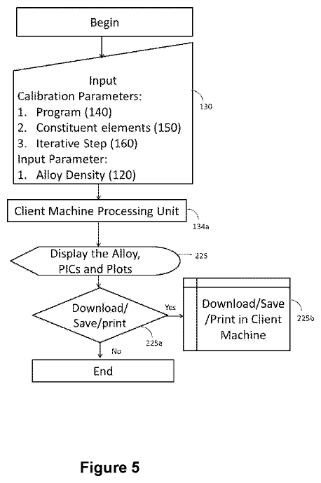

FIG. 5 illustrates the algorithm flow in the client machine input/output interface unit (130a) of a remote/server/cloud computing setup (137) for the DDS (100). The process begins with the input of calibration parameters (130), including the Metals program (140), constituent elements (150), and iterative step (160), as well as the input parameter, i.e., the alloy density (120) in input/output interface (130a). The data obtained from these inputs is then packed (134h) and transferred (134j) to the DDS (100). The client machine input/output interface unit (130a) receives the data packet (134k) from the DDS (100), unpacks the PICs and alloy composition data (134m), plots the visualizations (134n) enabling the user to analyze, explore the quantized e-Phantom database (170) for new alloys; and displays the alloy, PICs & plots (225), and download, save or print the results (225b) as per user's choice (225a).

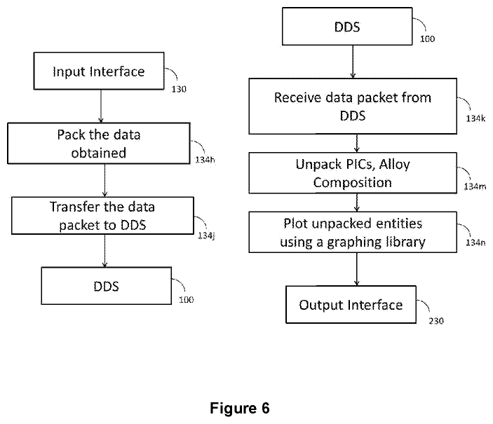

FIG. 6 depicts the algorithm flow in the client machine processing unit (134a) for a remote/server/cloud computing setup (137) for the DDS (100). The client machine processing unit (134a) receives the input and calibration parameters (130) from the client machine input/output interface unit (130a). It then packs the obtained data (134h) and transfers the data packet (134j) to the DDS (100). The DDS (100) processes the data and sends the results back to the client machine processing unit (134a), which receives the data packet (134k). The client machine processing unit (134a) then unpacks the PICs and alloy composition data (134m) and sends it to the client machine input/output interface unit (130a) for plotting (134n) and display (225) the output on interface (230).

The client machine input/output interface unit (130a) and client machine processing unit (134a) work together in the remote/server/cloud (137) computing setup for the DDS (100). The client machine input/output interface unit (130a) handles user inputs and displays the results, while the client machine processing unit (134a) facilitates the communication between the client machine input/output interface unit (130a) and the DDS (100) in remote/server/cloud (137) computing setup. The client machine processing unit (134a) sends the input data to the DDS (100) for processing and receives the results, which are then passed on to the client machine input/output interface unit (130a) for visualization (220) and user interaction.

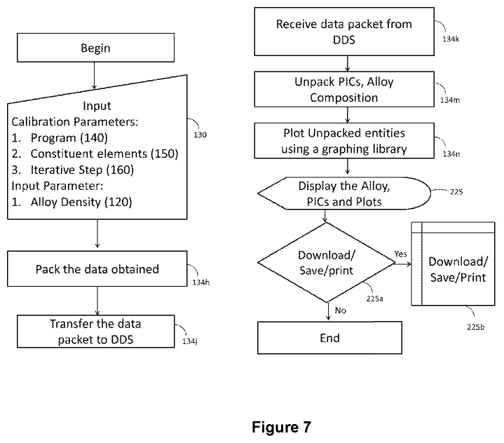

FIG. 7 depicts the algorithm flow in a stand-alone (135) computing setup for the DDS (100) according to an embodiment. The key steps include receiving the input parameters (130), packing the obtained data (134h), transferring the data packet (134j), transmitting them to the DDS (100) computing unit (165), receiving the computed data packet (134k), unpacking the PICs and alloy composition data (134m), plotting the visualizations (134n), enabling the user to analyze and explore the quantized e-Phantom database (170) for new alloys, displaying (225) the alloy, PICS & plots, and downloading, saving or printing the results (225b) as per user's choice (225a).

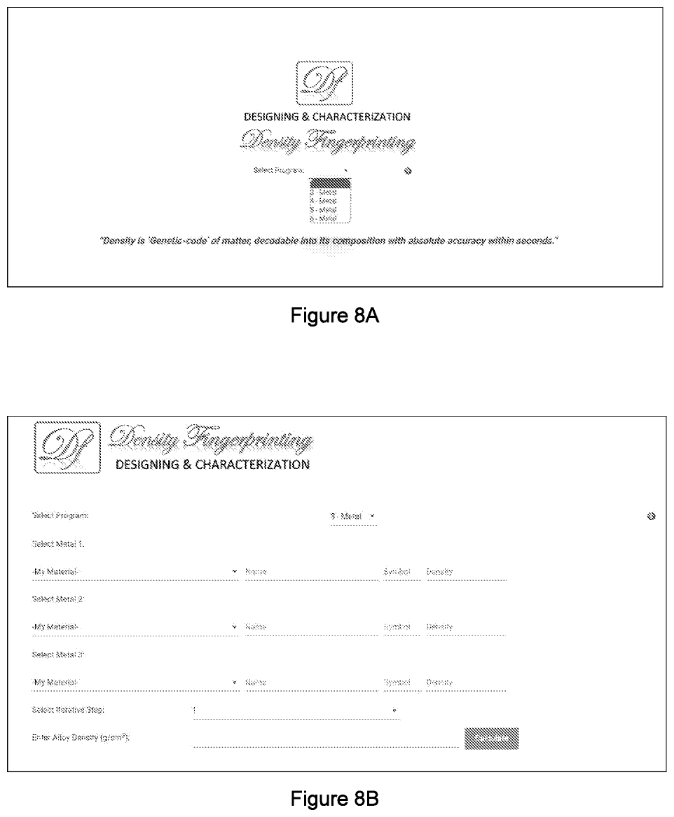

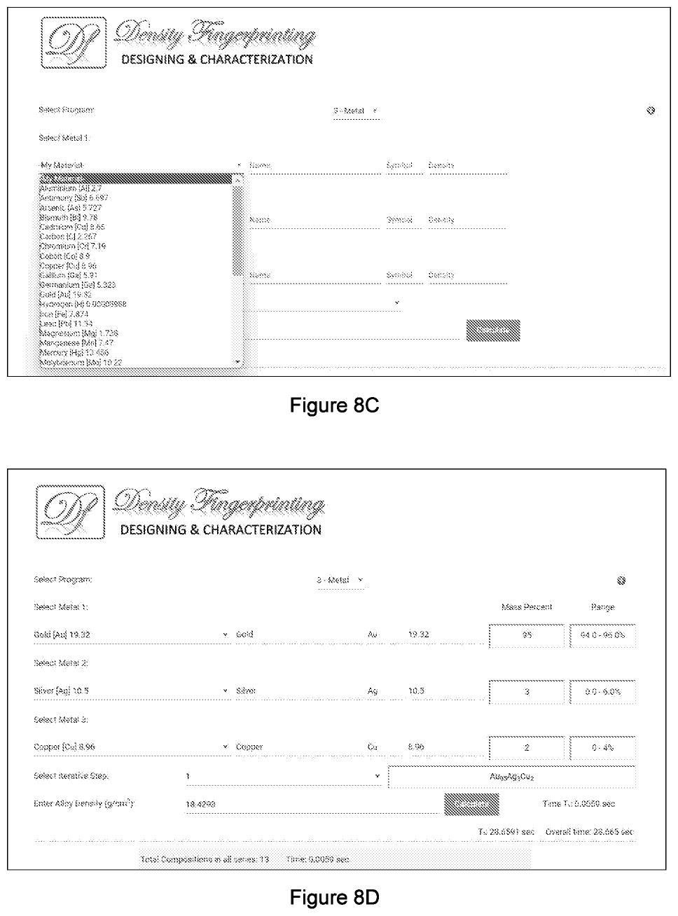

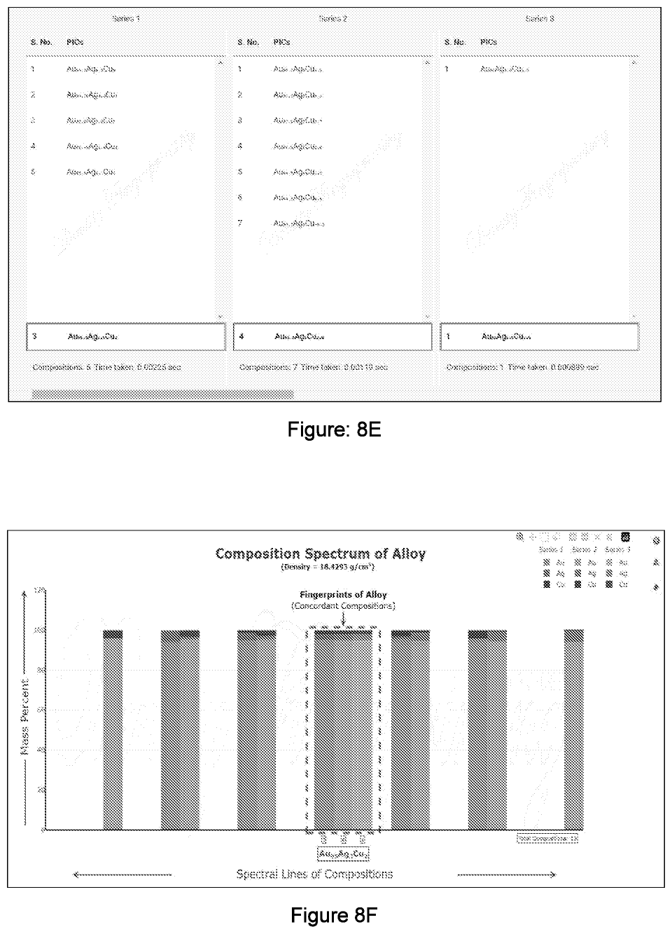

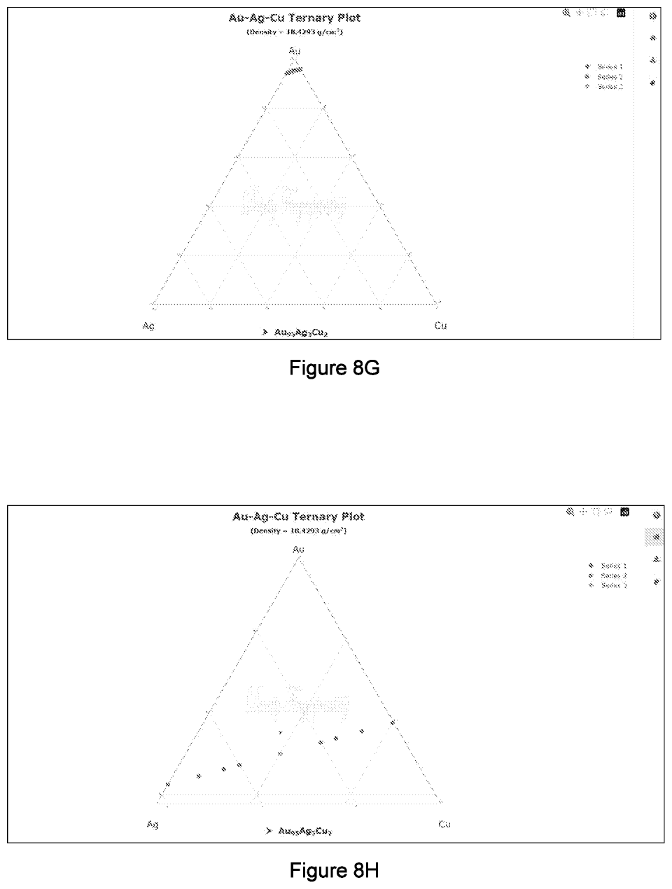

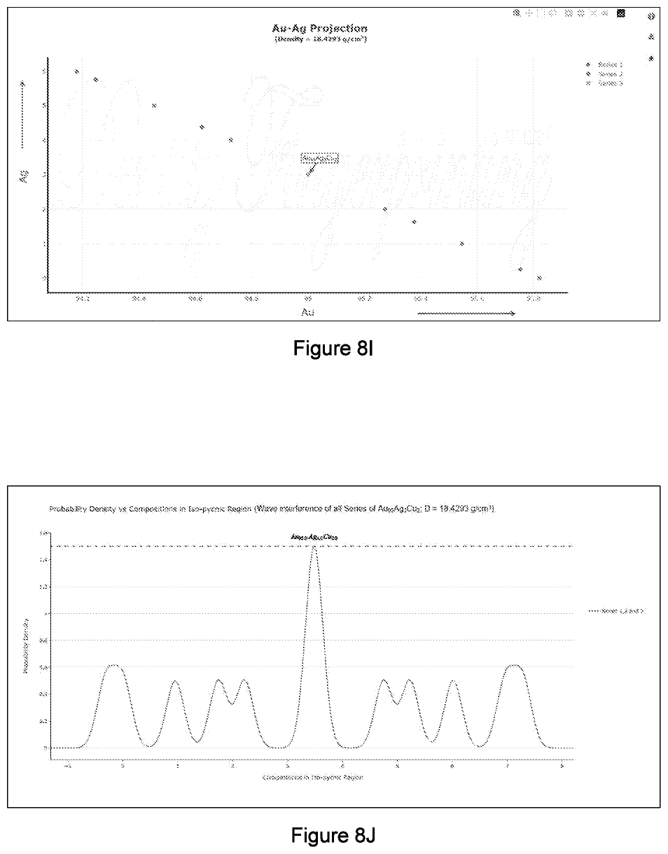

FIGS. 8A-J showcase various results and visualizations provided by the Density Decoding System (DDS) (100) for a ternary alloy (Au₉₅Ag₃Cu₂) example.

FIG. 8A illustrates the interface for selecting the desired Metals program from a drop-down menu.

FIG. 8B shows the interface for the 3-Metals program, where the user can select calibration parameters, including constituent elements (150) and the iterative step (default i=1) (160), and input the alloy density.

FIG. 8C demonstrates the selection of elements along with their standard densities from the drop-down menu in the 3-Metals program.

FIG. 8D displays the resultant True Composition (TC) of the alloy, along with its chemical formula (Au₉₅Ag₃Cu₂), percent range of elements (Au: 94-96%, Ag: 0-6%, Cu: 0-4%), and the total number of PICs (13) in all three PIC series for the input density of 18.4293. It also shows the execution time for displaying the resultant composition (0.0059 seconds) and data visualization (28.6591 seconds), with a total time of 28.665 seconds.

FIG. 8E displays all three series of Probable Iso-density Compositions (PICs) for the input density of 18.4293, along with their chemical formulae. The Concordant Compositions (CCs), identified as Au₉₅Ag₃Cu₂, are marked in red in each series.

FIG. 8F shows the composition spectrum constituting the fingerprints of the alloy Au₉₅Ag₃Cu₂ associated with the input density of 18.4293.

FIG. 8G illustrates the Isopycnic Region (IR) of all three PICs series associated with the density of 18.4293 in the Alloy Space (AS) within the Vast Alloy Space (VAS), constituting the Quantized Field of Compositions (QFCs) in the autopoietic e-Phantom database (170) in a 3-axis equilateral simplex.

FIG. 8H provides a magnified view of the three PICs series in the Isopycnic Region (IR), with the arrow-head indicating the point of merger of the CCs (Au₉₅Ag₃Cu₂), exhibiting superimposition, concordance, and vernier coincidence. FIG. 8I displays the Au—Ag 2D projection demonstrating the Isopycnic Region (IR) of the PICs series in the Alloy Space (AS) within the Vast Alloy Space (VAS). The arrow indicates the point of merger of the CCs (Au₉₅Ag₃Cu₂).

FIG. 8J shows the probability density vs. PICs series, demonstrating the wave interference of all series in the 3-Metals program, with the highest peak corresponding to the Most Probable Composition (MPC), i.e., Au₉₅Ag₃Cu₂, for the input density of 18.4293 in the Isopycnic Region (IR) of the Alloy Space (AS) within the Vast Alloy Space (VAS).

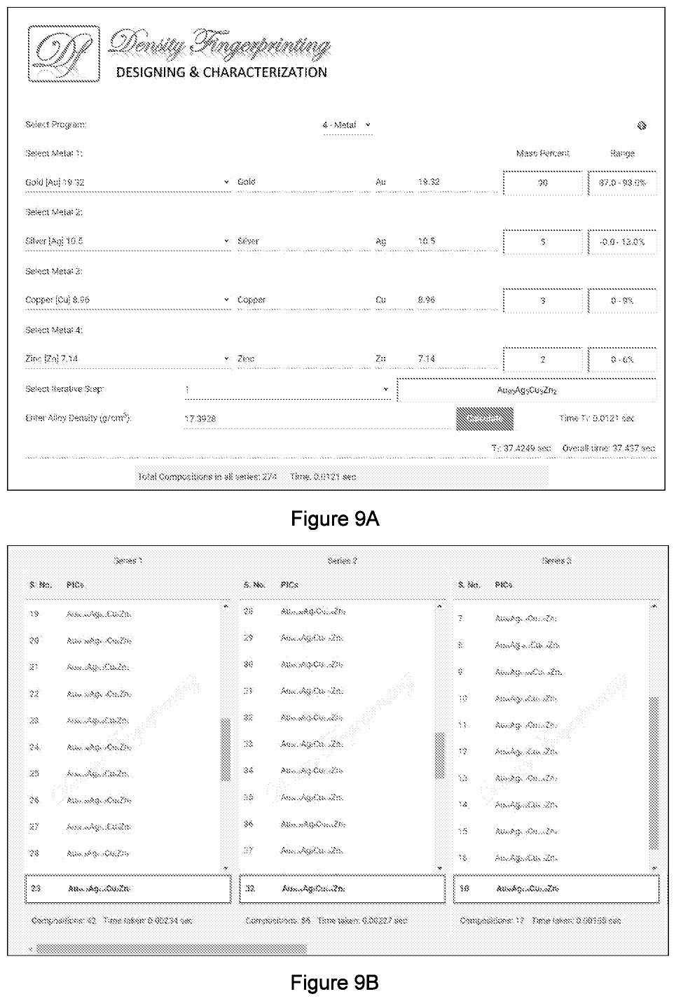

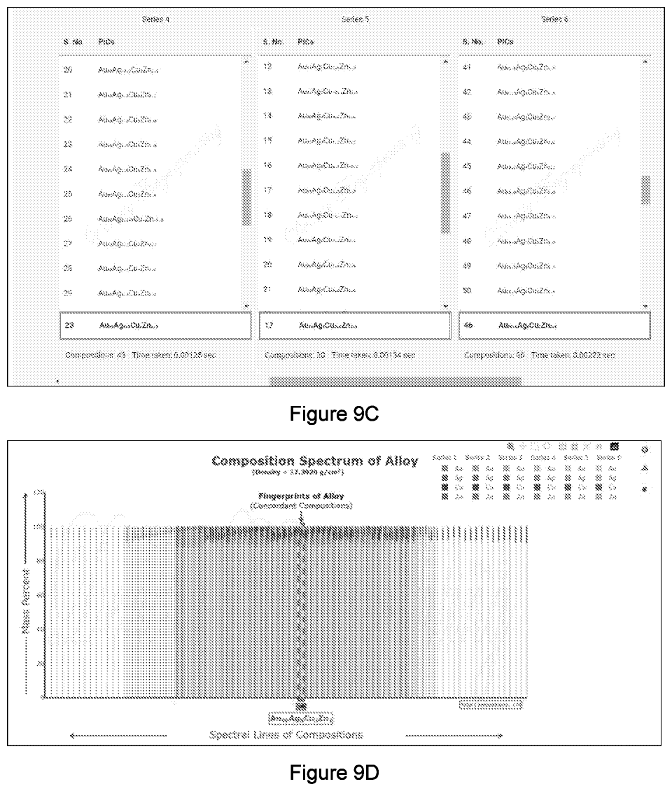

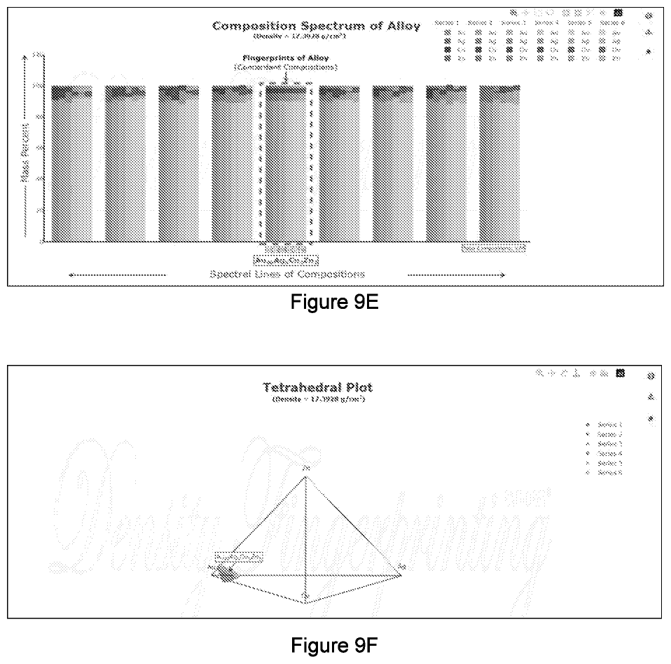

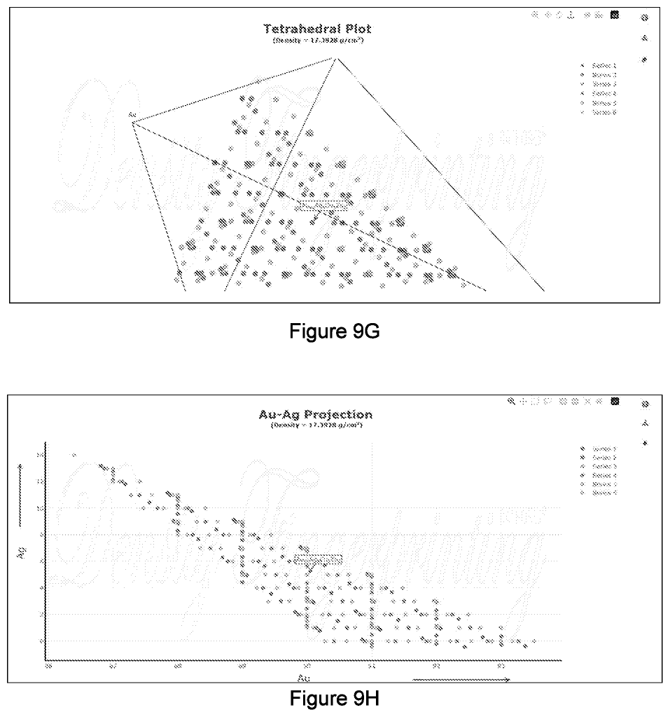

FIGS. 9A-I illustrate the results and visualizations for a quaternary alloy (Au₉₀Ag₅Cu₃Zn₂) example.

FIG. 9A displays the resultant True Composition (TC) of the alloy, along with its chemical formula (Au₉₀Ag₅Cu₃Zn₂), percent range of elements (Au (87-92%), Ag (0-12%), Cu (0-9%) and Zn (0-6%)), and the total number of PICs (274) in all six PIC series for the input density of 17.3928. It also shows the execution time for displaying the resultant composition (0.0121 seconds) and data visualization (37.4249 seconds), with a total time of 37.437 seconds.

FIGS. 9B and 9C displays all six series of Probable Iso-density Compositions (PICs) for the input density of 17.3928, along with their chemical formulae. The Concordant Compositions (CCs), identified as Au₉₀Ag₅Cu₃Zn₂, are marked in red in each series.

FIG. 9D presents the composition spectrum constituting asymmetric fractals as fingerprints of the alloy Au₉₀Ag₅Cu₃Zn₂.

FIG. 9E highlights the identical spectral lines of Concordant Compositions (CCs) of all the six series aligned together as a band, breaking asymmetry of the composition spectrum fractal to constitute the fingerprints of alloy Au₉₀Ag₅Cu₃Zn₂ associated with input density 17.3928. This underscores that any two PICs series are sufficient to obtain the fingerprints for conclusive identification of alloys which is crucial for alloys containing more than five elements due to geometric limitations in plotting higher-dimensional graphs. In such cases, 2D projections obtained from any two PICs series are sufficient to obtain fingerprints of higher order alloys.

FIG. 9F shows the Isopycnic Region (IR) of all six PICs series associated with the density of 17.3928 in the Alloy Space (AS) within Vast Alloy Space (VAS) constituting Quantised Field of Compositions (QFCs) in autopoietic e-Phantom database (170) in a 4-axis equilateral simplex.

FIG. 9G provides a magnified view of the six PICs series in the Isopycnic Region (IR), with the arrow indicating the point of merger of the CCs (Au₉₀Ag₅Cu₃Zn₂) exhibiting the superimposition, concordance and vernier coincidence.

FIG. 9H displays the Au—Ag 2D projection demonstrating the Isopycnic Region (IR) of the PICs series, with the arrow showing the point of merger of the CCs.

FIG. 9I presents the probability density vs. PICs series, demonstrating the wave interference of two series in the 4-Metals program, with the highest peak corresponding to the MPC (Au₉₀Ag₅Cu₃Zn₂).

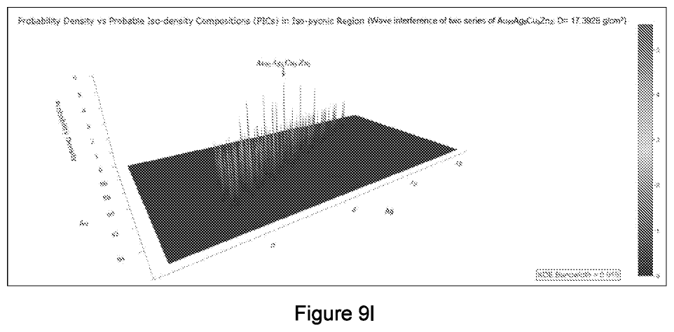

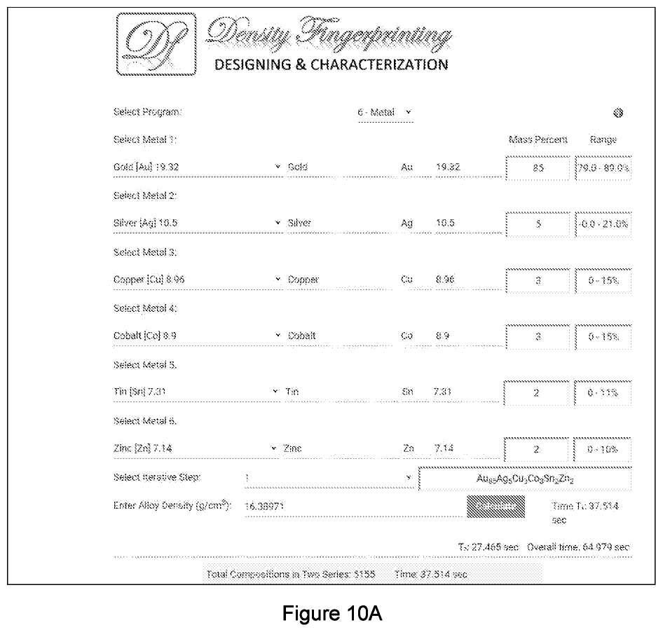

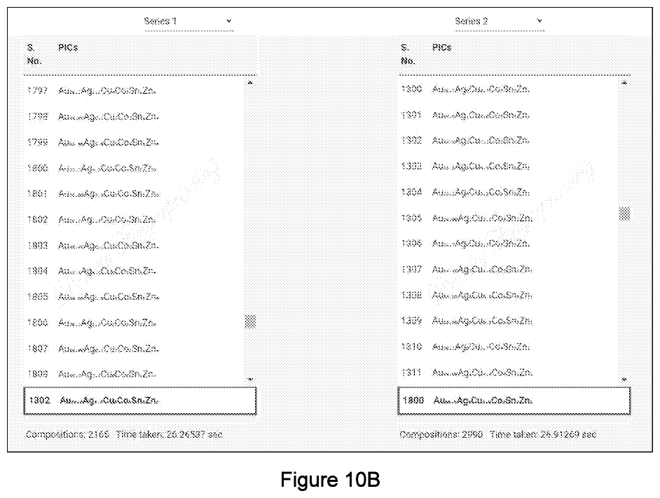

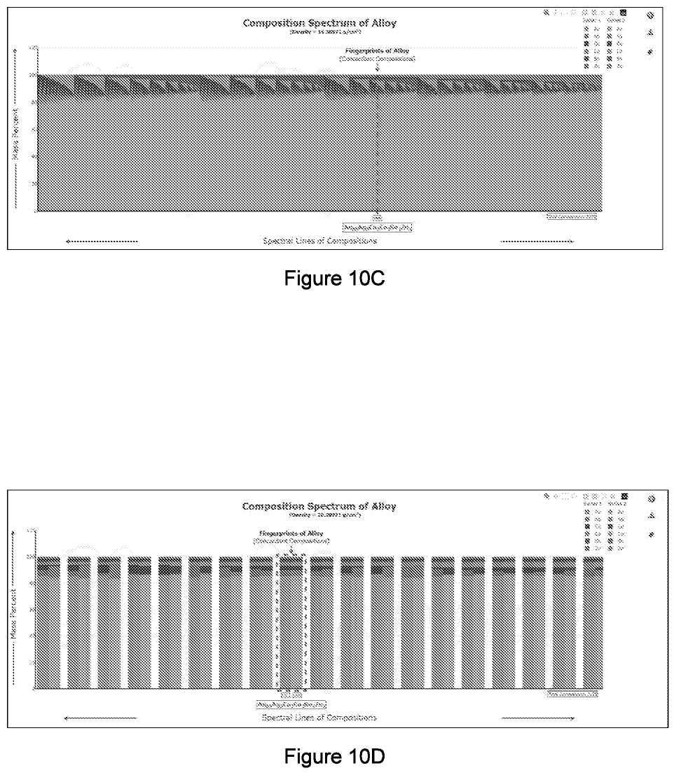

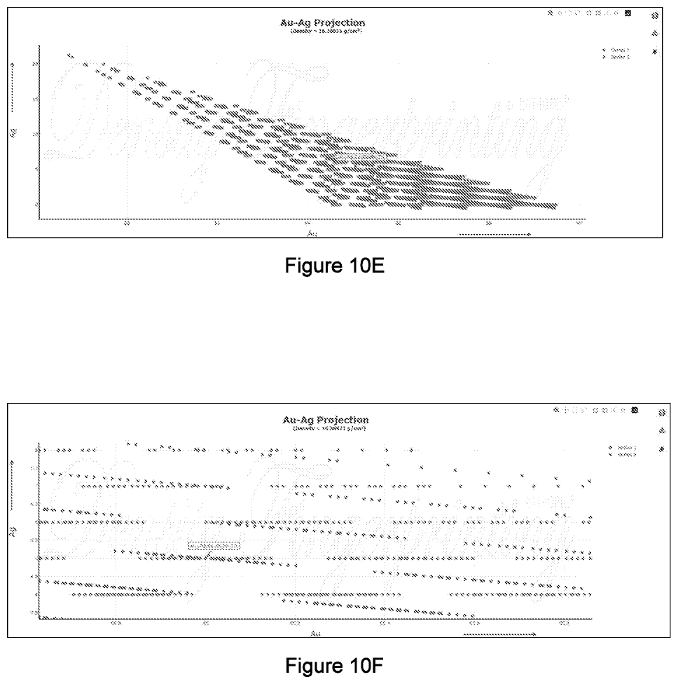

FIGS. 10A-F showcase the results and visualizations for a senary alloy (Au₈₅Ag₅Cu₃Co₃Sn₂Zn₂) example.

FIG. 10A displays the resultant True Composition (TC) of the alloy, along with its chemical formula (Au₈₅Ag₅Cu₃Co₃Sn₂Zn₂), percent range of elements (Au (79-89%), Ag (0-21%), Cu (0-15%), Co (0-15%) Sn (0-11) and Zn (0-10%)), and the total number of PICs (5155) or unique compositions in 2 out of 15 PIC series for the input density of 16.38971. It also shows the execution time for displaying the resultant composition (37.514 seconds) and data visualization (27.465 seconds), with a total time of 64.979 seconds.

FIG. 10B shows two series of Probable Iso-density Compositions (PICs) for the input density of 16.38971, along with their chemical formulae. The Concordant Compositions (CCs), identified as Au₈₅Ag₅Cu₃ Co₃Sn₂Zn₂, are marked in red in each series.

FIG. 10C presents the composition spectrum constituting asymmetric fractals as fingerprints of the alloy Au₈₅Ag₅Cu₃Co₃Sn₂Zn₂ associated with input density 16.38971.

FIG. 10D highlights the identical spectral lines of the CCs Au₈₅Ag₅Cu₃Co₃Sn₂Zn₂ from the two series aligned together as a band, breaking the asymmetry of the composition spectrum fractal.

FIGS. 10E and 10F display the Au—Ag 2D projection demonstrating the Isopycnic Region (IR) of the PICs series in the Alloy Space (AS) within Vast Alloy Space (VAS) constituting Quantised Field of Compositions (QFCs) in autopoietic e-Phantom database (170) in a 6-axis equilateral simplex, with the arrow showing the point of merger of the CCs (Au₈₅Ag₅Cu₃Co₃Sn₂Zn₂).

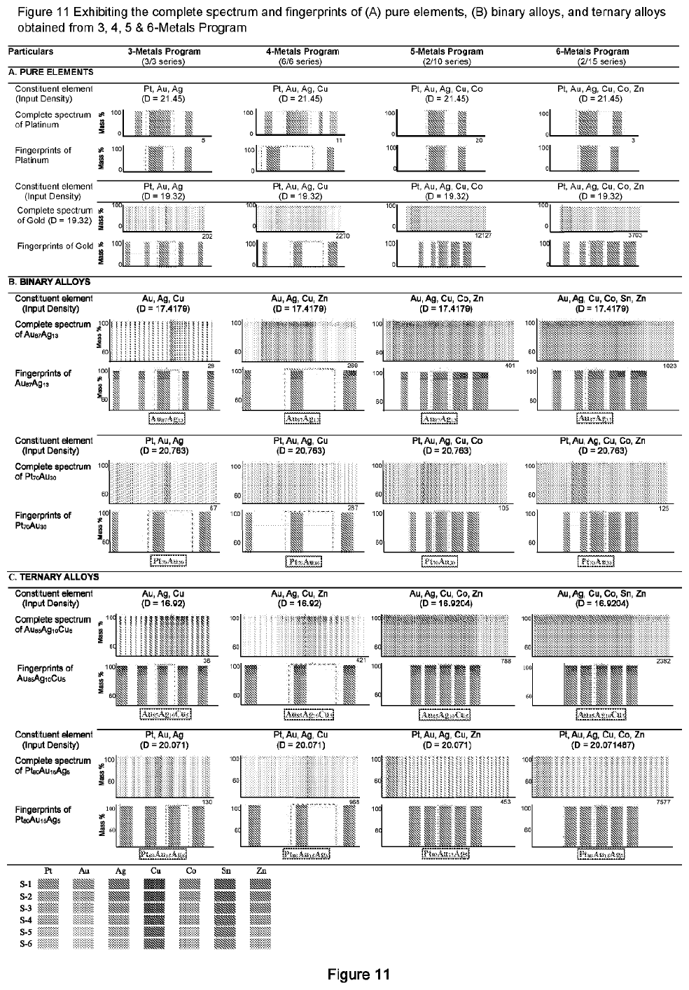

FIG. 11 exhibits the complete spectrum and fingerprints of (A) pure elements Pt & Au, (B) binary alloys Au₈₇Ag₁₃ & Pt₇₀Au₃₀, and (C) ternary alloys Au₈₅Ag₁₀Cu₅ & Pt₈₀Au₁₅Ag₅ obtained from the 3, 4, 5, and 6-Metals Programs.

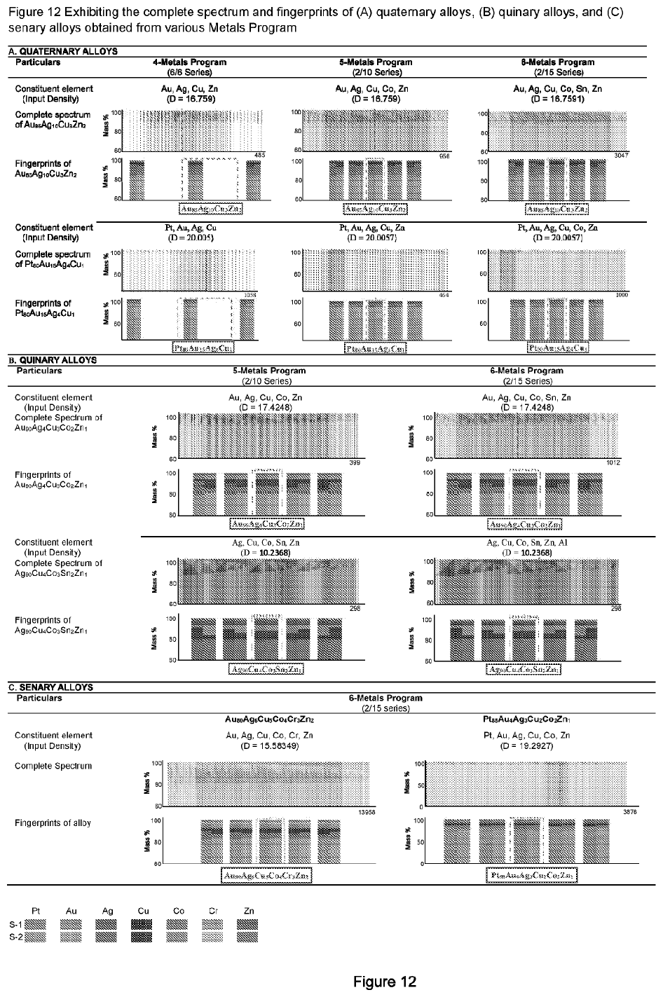

FIG. 12 exhibits the complete spectrum and fingerprints of (A) quaternary alloys Au₈₅Ag₁₀Cu₃Zn₂ & Pt₈₀Au₁₅Ag₄Cu₁, (B) quinary alloys Au₉₀Ag₄Cu₃Co₂Zn₁ & Ag₉₀ Cu₄Co₃Sn₂Zn₁, and (C) senary alloys Au₉₀Ag₆Cu₅Co₄Cr₃Zn₂ & Pt₈₈Au₄Ag₃Cu₂Co₂Zn₁ obtained from corresponding Metals Programs.

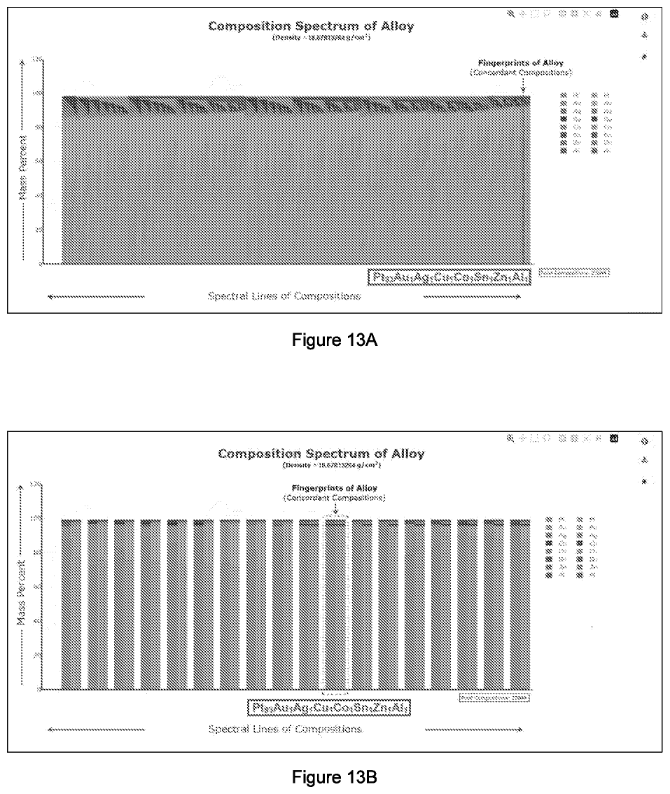

FIGS. 13A and 13B illustrate the results for an octonary alloy (Pt₉₃Au₁Ag₁Cu₁Co₁Sn₁Zn₁Al₁) example with an input density of 18.67813264.

FIG. 13A displays the composition spectrum of 2 out of 28 PIC series containing 27044 PICs or unique compositions constituting asymmetric fractals as fingerprints of the alloy Pt₉₃Au₁Ag₁Cu₁Co₁Sn₁Zn₁Al₁.

FIG. 13B shows the identical spectral lines of the CCs Pt₉₃Au₁Ag₁Cu₁Co₁Sn₁Zn₁Al₁ from the 2 out of 28 PIC series aligned together as a band, breaking the asymmetry of the composition spectrum fractal.

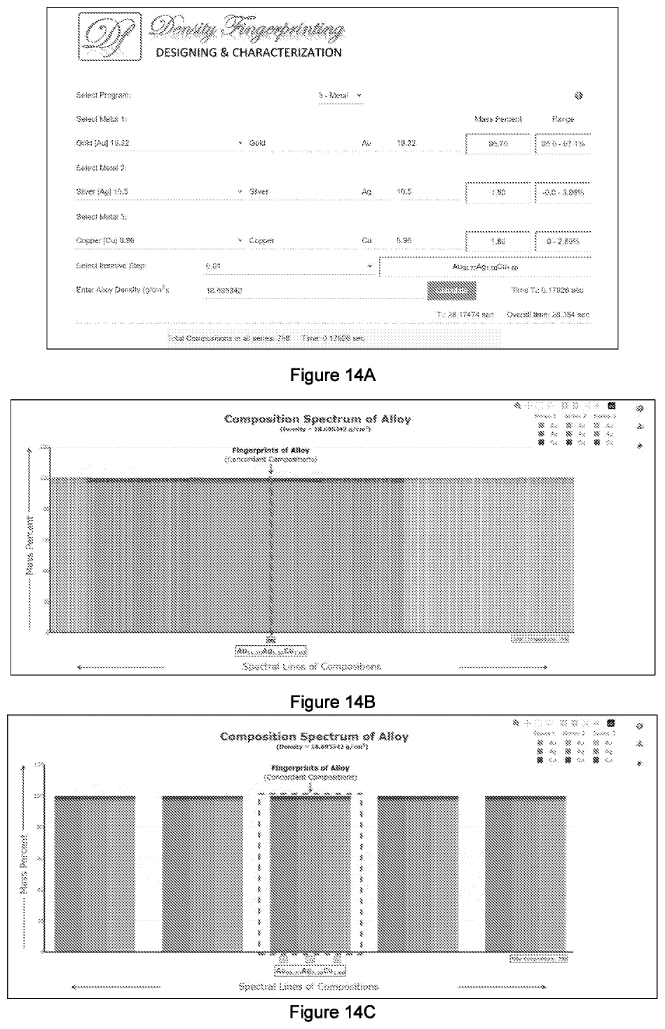

FIGS. 14A-C illustrate the results for a ternary alloy (Au_{96.7}Ag_{1.5}Cu_{1.8}) example with an input density of 18.695342.

FIG. 14A displays the resultant True Composition (TC) of the alloy, along with its chemical formula (Au_{96.7}Ag_{1.5}Cu_{1.8}), range of Au (96-97.1%), Ag (0-3.98%) and Cu (0-2.89%), and the total number of PICs (798) in three PIC series for the input density of 18.695342 at iterative step i=0.01. It also shows the overall execution time for displaying the resultant composition and data visualization as 28.354 seconds.

FIG. 14B displays the composition spectrum of three PIC series containing 798 PICs constituting asymmetric fractals as fingerprints of the alloy Au_{96.7}Ag_{1.5}Cu_{1.8}.

FIG. 14C shows the identical spectral lines of the CCs Au_{96.7}Ag_{1.5}Cu_{1.8} from the three PIC series aligned together as a band, breaking the asymmetry of the composition spectrum fractal.

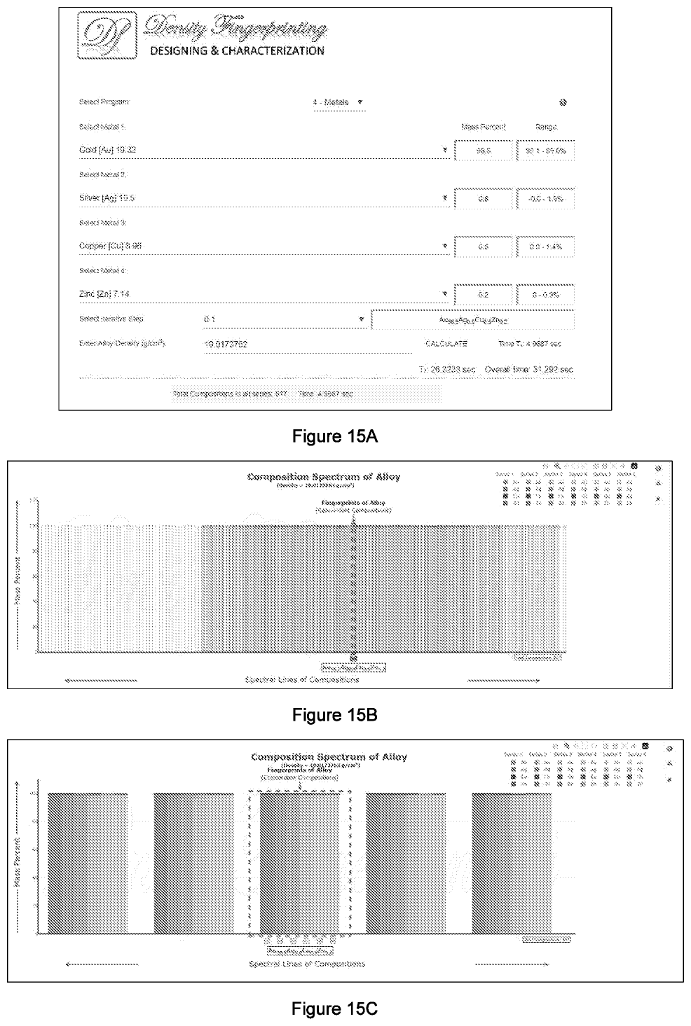

FIGS. 15A-C illustrate the DDS (100) interfaces showcasing the composition spectrum and fingerprints for a quaternary alloy example.

FIG. 15A displays the resultant True Composition (TC) of the alloy, along with its chemical formula (Au_{98.5}Ag_{0.8}Cu_{0.5}Zn_{0.2}), range of Au (98.1-99.0%), Ag (0-1.9%), Cu (0-1.4%) and Zn (0-0.9%), and the total number of PICs (517) in six series for the input density of 19.0173762 at iterative step i=0.1. It also shows the overall execution time for displaying the resultant composition and data visualization as 31.292 seconds.

FIG. 15B displays the composition spectrum of six PIC series containing 517 PICs constituting asymmetric fractals as fingerprints of the alloy Au_{98.5}Ag_{0.8}Cu_{0.5}Zn_{0.2}.

FIG. 15C shows the identical spectral lines of the CCs Au_{98.5}Ag_{0.8} Cu_{0.5}Zn_{0.2} from the six PIC series aligned together as a band, breaking the asymmetry of the composition spectrum fractal.

The modified Archimedes density equations enabling the inclusion of additional constituent elements are:

$$\begin{matrix}{m}_{1} = \frac{[ [ ( 100 - {\sum }_{i = 3}^{n} {m}_{i} ) * ( \frac{1}{{d}_{2}} ) ] - [ ( \frac{100}{D} ) - {\sum }_{i = 3}^{n} ( \frac{{m}_{i}}{{d}_{i}} ) ] ]}{( \frac{1}{{d}_{2}} - \frac{1}{{d}_{1}} )} \\ {m}_{2} = 100 - [ {\sum }_{i = 3}^{n} {m}_{i} ]\end{matrix}$$

where,
   D=density of the alloy in g/cm³,
   m₁, m₂, m₃ . . . mₙ=mass of constituent elements,
   d₁, d₂, d₃ . . . dₙ=standard densities of constituent elements in g/cm³ (arranged in order d₁>d₂>d₃> . . . dₙ), and
   n=number of constituent elements.

The PICs are computed by iteratively feeding SPIN-values for the additional constituent elements into these equations. This allows discretizing the infinite PICs into a quantized finite set. Each PIC series computes a unique set of compositions for a given density based on unique SPIN-value combinations.

The compiler module (180) compiles the computed PICs into respective PIC series to constitute a real-time database (i-Database) (185) for the input density (120), and maps the PIC series in the Quantized Field of Compositions (QFCs) to visualize them as Isopycnic Regions (IRs) in the Alloy Space (AS) of the Vast Alloy Space (VAS) in the e-Phantom database (170).

Entire space occupied by the potential alloys constituting Quantized Field of Compositions (QFCs) in e-Phantom database (170) within n-axis equilateral simplex of selected constituent elements (150) is characterised as the Vast Alloy Space (VAS). The iso-density pocket created due to Probable Iso-density Compositions (PICs) constitutes Isopycnic Region (IR) in Vast Alloy Space (VAS). It is regarded as Alloy Space (AS), which specifies the space occupied by IR of particular density (120), where there is maximum probability of finding composition of alloy associated with such input density.

The analyzer module (190) identifies the Concordant Compositions (CCs) among the PIC series as the Most Probable Composition (MPC). However, the MPC only represents the True Composition (TC) of the alloy if its density matches the effective density constituting the Density Genome (DG) of the corresponding compositional pocket in the Quantized Field of Compositions (QFCs) of the Vast Alloy Space (VAS) in the e-Phantom database (170).

The selection of n-elements Program (140) in DDS (100) constitutes an n-axis equilateral simplex grid, featuring a percent grid (p-Grid) graduated with 0-100 equal divisions on each axis, representing percent fractions of variables selected at vertices. The p-Grid's least count is directly tied with an infinitesimally reducible Iterative-step (160), which, in turn, regulates the accuracy level of the p-Grid. This p-grid serves as foundation for the quantized-pockets, ranging from whole numbers to infinitesimal accuracy levels from i=1 to i→0.

The quantised-pockets in the continuum of p-Grid represent the pocket-points, each pocket-point holding the value of proportional percent fractions of variables placed on vertices by virtue of its position is regarded as percent-pocket point (p-Pocket points).

On selecting the constituent elements (150) in the DDS (100), their standard densities occupy the vertices of p-Grid creating a Secondary-grid, the Density Grid (d-Grid) alongside the p-Grid of the simplex. The continuum of d-Grid comprises an infinite number of density points, each point precisely representing the proportional ratio of standard densities of selected elements. The d-Grid constitutes a single dimension grid between the constituent elements (150) of lowest and highest densities.

Notably, both the grids (i.e., p & d-Grids) commonly share the same continuum in the n-axis equilateral simplex, where each density point of the d-Grid eclipses and superimposes the p-Pocket point. The interaction between the intrinsic ratios of the standard densities of selected elements in the density points of the d-Grid and the percentage fractions in the p-Pocket points of the p-Grid ultimately results in an instantaneous transformation of the density points into their elemental percent compositions.

This interaction creates a Quantized Field of Compositions (QFCs) of alloys, transforming the entire continuum or Vast Alloy Space (VAS) of the n-axis equilateral simplex into an exhaustive Phantom database (or e-Phantom database) (170). This autopoietic e-Phantom database (170) of all potential alloys of selected constituent elements (150) is instantaneously established within the Vast Alloy Space (VAS) as soon as the constituent elements (150) are selected in the p-Grid of the system.

The selection of the desired accuracy level through the Iterative-step (i) facilitates precise demarcation and visualization of intended accuracy levels of compositions in QFCs of Vast Alloy Space in e-Phantom database (170) with absolute precision and accuracy.

At the initial accuracy level (i.e., i=1), the p-Grid visualizes all the pockets containing only whole number compositions in e-Phantom database (170), whereas the reduced iterative steps i.e., i=0.1, 0.01, 0.001, and so on visualise the pockets containing fractional compositions of corresponding accuracies, such as 1st, 2nd, 3^{rd} and so forth decimal places respectively.

The p-Grid invariably accommodates a unique and finite number of compositions at each accuracy level of the iterative step. With each reduction in the iterative step, the parent pockets undergo repeated segmentation into smaller nested pockets of higher accuracies holding unique alloys of corresponding higher accuracies.

This segmentation extends infinitesimally with each reducing iterative step (i.e., i=1 to i→0) unless singularity of segmented micro-pockets is achieved, where each pocket holds single density of a distinct composition at lowest iterative step representing the highest accuracy level.

Thus, the Quantised Field of Compositions (QFCs) of Vast Alloy Space (VAS) of entire e-Phantom database (170) created in the continuum of n-axis equilateral simplex grid perfectly holds all potential compositions of constituent element selected in DDS (100).

The Iterative step (i) perfectly segments the entire VAS into distinct levels of accuracies, each level holding finite number of pockets, and each pocket holding unique composition of alloy of selected elements at such accuracy level as selected through the Iterative step. We have conclusively determined the finite number of pockets associated with different accuracy levels at varying iterative steps, where each pocket holds unique alloy of corresponding accuracy level.

The autopoietic e-Phantom database (170) is generated instantaneously upon selecting the constituent elements (150). It consists of a percent grid (p-Grid) defining the composition pockets of all potential alloy compositions of selected constituent elements (150) and a density grid (d-Grid) of the corresponding standard densities ranging from lowest density to highest density. Each effective density is a Density Genome (DG) encoding the True Composition (TC) for its pocket.

To conclusively determine the True Composition (TC) of the alloy, the analyzer module (190) maps the MPC density to the DGs at the selected iterative step (160). If a match is found, the MPC is confirmed as the TC. If not, the iterative step (160) is refined (195) to increase the resolution of the QFCs pockets and the process repeats until the MPC matches a DG.

The analyzer module (190) also mines the QFCs to identify composition pockets that are not represented in existing alloy databases/libraries. These unexplored pockets represent potential new alloys. The DDS (100) highlights these pockets in the output visualizations, guiding the purposeful design and discovery of new alloys. Specific alloy compositions can be zoomed into and their properties predicted based on machine learning models trained on existing alloy data.

This density fingerprinting approach enables the DDS (100) to accurately decode the True Composition (TC) of any multi-component alloy from its density alone. The PIC series generate unique density fingerprints visualized as spectral patterns with the Concordant Compositions (CCs) disrupting the asymmetry of their fractals.

To ensure the accurate functioning of the DDS (100), a calibration mechanism is incorporated in calibration module. This mechanism allows the user to constitute imaginary alloys with a desired number of constituent elements (150) and calculate their Archimedean densities using the equation:

$$D = \frac{M}{V} = \frac{[ {m}_{1} + {m}_{2} + {m}_{3} + … + {m}_{n} ]}{[ ( \frac{{m}_{1}}{{d}_{1}} ) + ( \frac{{m}_{2}}{{d}_{2}} ) + ( \frac{{m}_{3}}{{d}_{3}} ) + … + ( \frac{{m}_{n}}{{d}_{n}} ) ]}$$

where,
   D=density of the alloy in g/cm³,
   M=mass of the alloy (100 units),
   m₁, m₂, m₃ . . . mₙ=mass of constituent elements,
   d₁, d₂, d₃ . . . dₙ=standard densities of constituent elements in g/cm³, and
   n=number of constituent elements.
      The calculated densities of these known imaginary alloys, referred to as Archimedean alloys, are then subjected to the DDS (100) as input parameters. By observing whether the DDS (100) correctly decodes the compositions of these Archimedean alloys, the accurate functioning of the system can be authentically verified. This calibration process ensures the reliability and trustworthiness of the DDS (100) in determining the elemental compositions of real-world alloys.

The computer-implemented method for determining elemental percent compositions of alloys from their densities involves the following steps:
(a) receiving, via an input interface (130, 130a), an input density (120) of an unknown alloy (105) and calibration parameters;
(b) computing, using a processor (134, 134a) executing a mathematical module (Maths-mill) (175), PICs of the unknown alloy (105) using modified Archimedes density equations;
(c) compiling, using the processor executing a compiler module (180), the computed PICs into respective PIC series to constitute a real-time database (185) and mapping the PIC series in the QFCs in an autopoietic e-Phantom database (170);
(d) identifying, using the processor executing an analyzer module (190), CCs among the PIC series as a Most Probable Composition (MPC);
(e) mapping the MPC to effective densities in the QFCs pockets;
(f) iteratively refining (195) the MPC until convergence to determine the True Composition (TC); and
(g) outputting, via an output interface (230, 130a), the determined True Composition (TC) of unknown alloy (105) as characterisation (210), alloy designing data (200), and visualizations (220).

The computer system for determining elemental percent compositions of alloys from their densities comprises:
(a) a processor (134, 134a);
(b) an input interface (130, 130a) coupled to the processor (134, 134a) for receiving input parameters;
(c) an output interface (230, 130a) coupled to the processor (134, 134a) for displaying (225) results; and
(d) a memory (133, 133a) coupled to the processor (134, 134a), storing instructions which, when executed by the processor, cause the system to:
   compute PICs of the alloy (105) using modified Archimedes density equations;
   compile PIC series and map them in the QFCs in an autopoietic e-Phantom database (170);
   identify CCs among the PIC series as a Most Probable Composition (MPC);
   map the MPC to effective densities in the QFCs pockets;
   iteratively refine (195) the MPC until convergence to determine the True Composition (TC); and
   output the determined True Composition (TC) of unknown alloy (105) as characterisation (210), alloy designing data (200), and visualizations (220).

The computer program product for determining elemental percent compositions of alloys from their densities comprises a non-transitory computer readable medium (133, 133a) having program instructions embodied therewith. The program instructions are executable by a processor (134, 134a) to cause the processor to perform a method comprising:
(a) receiving an input density (120) of an unknown alloy (105) and calibration parameters;
(b) computing PICs of the unknown alloy (105) using modified Archimedes density equations;
(c) compiling the computed PICs into respective PIC series to constitute a real-time database (185) and mapping the PIC series in the QFCs in an autopoietic e-Phantom database (170);
(d) identifying CCs among the PIC series as a Most Probable Composition (MPC);
(e) mapping the MPC to effective densities in the QFCs pockets;
(f) iteratively refining (195) the MPC until convergence to determine the True Composition (TC) of unknown alloy (105); and
(g) outputting the determined True Composition (TC) of unknown alloy (105) as characterisation (210), alloy designing data (200), and visualizations (220).
   The program instructions may further cause the processor (134, 134a) to:
(a) select a target density;
(b) provide a target value or range for the density;
(c) analyze the autopoietic e-Phantom database (170) to identify composition pockets that exhibit the target density value or range;
(d) extract the elemental percent ranges associated with the identified composition pockets; and
(e) design an alloy (200) by selecting elemental percentages within the extracted ranges.
   Additionally, the program instructions may cause the processor (134, 134a) to:
(a) analyze the autopoietic e-Phantom database (170) to identify composition pockets that exhibit uncommon density values;
(b) extract the elemental percent ranges associated with the identified composition pockets;
(c) design a set of candidate alloys (200) by selecting elemental percentages within the extracted ranges; and
(d) synthesize and test the candidate alloys to validate their densities.

The DDS (100) is highly ergonomic and cost-effective compared to existing techniques. It requires only density as an input (120) and can handle alloys with any number of constituent elements (150). The decoded compositions have been validated with absolute accuracy for various binary, ternary, and higher-order alloys.

The DDS (100) enables rapid, non-destructive determination of alloy compositions, design of alloys (200) with targeted densities, discovery of new alloys with unique density profiles, optimization of alloys for specific density-critical applications and design of gradient alloys.

The DDS (100) demonstrates a hierarchical relationship among its various metals programs (140), with each higher metals program encompassing and substituting the functions of the lower metals programs (140). Each program exhibits proficiency in decoding input densities (120) across the entire spectrum of the e-Phantom database (170), ranging from the lowest to the highest densities of the selected constituent elements (150).

The DDS (100) initiates each program with the assumption that the input density (120) may be associated with any conceivable combination of selected constituent elements (150). Consequently, it conducts a comprehensive procedural analysis for each input density (120), aiming to determine the true outcome, regardless of a perfect match with the standard density of the selected constituent elements (150). The constituent elements associated with the input density (120) form a subset of the selected constituent elements (150) in the program (140). Any selected constituent elements (150) not present among the constituent elements in the alloy associated with the input density (120) is automatically zeroed out from the Concordant Compositions (CCs), reflecting in the end result.

This novel approach enables the DDS (100) to determine the constitution of all alloys, including binaries having singular compositions without series, as well as every individual pre-selected metal having no composition at all. Densities of non-binary alloys are always associated with three or more series of Probable Iso-density Compositions (PICs), as per combinatorial notation ⁿC₂, and these series always have one common PIC exhibiting concordant compositions. This discovery made it possible to find out the Most Probable Compositions (MPCs) for densities of alloys and to conclusively determine the True Composition (TC) by mapping the MPCs in the e-Phantom database (170) pockets for effective densities constituting the Density Genome (DG).

The Density Decoding System (DDS) (100) and its associated methods can be effectively utilized for designing alloys (200) with targeted densities (120). The method involves selecting a target density (120), providing a target value or range for the density, analyzing the autopoietic e-Phantom database (170) to identify composition pockets that exhibit the target density value or range, extracting the elemental percent ranges associated with the identified composition pockets, and designing an alloy (200) by selecting elemental percentages within the extracted ranges. This approach enables the rational design of alloys (200) with desired densities, streamlining the alloy development process.

To further optimize the alloy design (200) process, the designed alloy can be synthesized, and its actual density can be measured. By comparing the actual density with the target value or range, the alloy design (200) can be iteratively refined (195) until the desired density is achieved. This feedback loop ensures that the final alloy meets the specified density requirements.

The DDS (100) and its methods also enable the discovery of new alloys with unique density profiles. By analyzing the autopoietic e-Phantom database (170), composition pockets that exhibit uncommon density values can be identified. The elemental percent ranges associated with these composition pockets are extracted, and a set of candidate alloys is designed (200) by selecting elemental percentages within the extracted ranges. The candidate alloys are then synthesized and tested to validate their densities, leading to the discovery of novel alloys with unprecedented density profiles.

To gain deeper insights into the structure-density relationships of the validated alloys, their microstructure and phase composition can be characterized. By correlating the microstructure and phase composition with the measured densities, the alloy design (200) can be further refined (195) to optimize the desired density. This approach enables the rational design of alloys (200) with tailored microstructures and densities.

The DDS (100) and its methods can also be applied to optimize alloy compositions for specific density-critical applications. By providing a set of application-specific density requirements, the autopoietic e-Phantom database (170) can be analyzed to identify composition pockets that satisfy these requirements. The elemental percent ranges associated with the identified composition pockets are extracted, and a set of candidate alloys is designed (200) by selecting elemental percentages within the extracted ranges. The candidate alloys are then ranked based on their predicted density performance, cost, and manufacturability, and the top-ranked alloys are selected for further development and testing. This approach enables the optimization of alloy compositions for specific density-critical applications.

The Density Decoding System (DDS) (100) and its associated methods can be extended to the development of gradient alloys with spatially varying densities. Gradient alloys are materials with compositional and property gradients that are tailored for specific applications, such as aerospace components, medical implants, and energy storage devices. The DDS (100) enables the design (200) of gradient alloys by leveraging the autopoietic e-Phantom database (170) and the density fingerprinting approach. By selecting a set of target densities (120) that vary spatially according to application requirements and analyzing the e-Phantom database (170), composition pockets that exhibit the desired spatial variation of densities can be identified. The elemental percent ranges associated with these composition pockets are extracted, and a gradient alloy is designed by selecting elemental percentages within the extracted ranges and specifying their spatial distribution. The gradient alloy can then be synthesized using additive manufacturing or other suitable techniques, such as powder metallurgy or laser cladding. The DDS (100) streamlines the gradient alloy development process by enabling the rapid identification of composition ranges that satisfy the desired spatial variation of densities, reducing the need for extensive experimental iterations. Furthermore, the DDS (100) can potentially lead to the discovery of novel gradient alloys with unique combinations of properties, expanding the design space for advanced materials.

In summary, the DDS (100) and its density fingerprinting approach pioneered in this invention transform the classical Archimedes method into a powerful technique for non-destructive compositional analysis and inverse design of complex alloys. By synergistically combining physical laws with computational methods, this innovation unlocks the full potential of density as a fundamental descriptor. It sets the stage for the accelerated discovery of new alloys with unique density profiles for a sustainable future.

### EXAMPLES

### Example-1

The density of imaginary ternary alloy Au_{96.7}Ag_{1.5}Cu_{1.8} calculated using the calibration module of Density Decoding System (DDS) (100) was found D=18.695342. It was used to scrutinise and ascertain overall function and working efficiency of DDS.

The following results were obtained on subjecting this density in DDS (100) as input parameter:
In first step, we configured the DDS to show results obtained at each iterative step (i). The DDS (100) demonstrated,
(a) at iterative step i=1, DDS (100) generated 11 PICs in 3 series within 23.256 sec. showing the presence of a binary alloy Au₉₆Ag₄ as MPC-1
(b) at iterative step i=0.1, DDS (100) generated 82 PICs in 3 series within 23.548 sec. showing the presence a ternary alloy Au_{96.7}Ag_{1.5}Cu_{1.8} as MPC-2
(c) at iterative step i=0.01, DDS (100) generated 798 PICs in 3 series within 28.354 sec. repeatedly showing the presence of identical ternary alloy Au_{96.70}Ag_{1.50}Cu_{1.80} as
MPC-3.
Remarkably, MPC-3 being equal to MPC-2 exhibited convergence showing conclusively confirmed True Composition (TC) of alloy as Au_{96.70}Ag_{1.50}Cu_{1.80} associated with input density D=18.695342.
   In second step, we configured the DDS (100) to show overall final result in one go. The DDS (100) conclusively determined the True Composition (TC) of alloy Au_{96.70}Ag_{1.50}Cu_{1.80} as converged MPC-3 after performing all the three successive steps of decoding the input density of unknown alloy within 28.378 sec.
   The DDS (100) displayed the presence of 798 unique compositions (PICs) of alloys in Isopycnic Region (IR) at iterative step i=0.01 in the Alloy Space (AS) within the Vast Alloy Space (VAS), constituting the Quantized Field of Compositions (QFCs) in the autopoietic e-Phantom database (170). The DDS (100) also displayed percent ranges of constituent elements (150) in these alloys at this accuracy level being Au (96-97.1%), Ag (0-3.98%) and Cu (0-2.89%).
   The interface of DDS (100), composition spectrum and fingerprints of this alloy are illustrated in FIGS. 14A, B & C.

### Example-2

Similarly, the density of imaginary gold alloy Au_{98.5}Ag_{0.8}Cu_{0.5}Zn_{0.2} containing Ag Cu and Zn as trace metals calculated using the calibration module of Density Decoding System (DDS) (100) was found D=19.0173762. It was used to scrutinise and ascertain overall function and working efficiency of DDS (100).

The following results were obtained on subjecting this density in DDS (100) as input parameter:
In first step, we configured the DDS (100) to show results obtained at each iterative step (i). The DDS demonstrated,
(a) at iterative step i=1, DDS (100) generated 20 PICs in 6 series within 24.3126 sec. showing the presence of a binary alloy Au₉₈Ag₂ as MPC-1
(b) at iterative step i=0.1, DDS (100) generated 517 PICs in 6 series within 31.2920 sec. showing the presence a quaternary alloy Au_{98.5}Ag_{0.8} Cu_{0.5}Zn_{0.2} as MPC-2
(c) at iterative step i=0.01, DDS (100) generated 42500 PICs in 6 series within 63.6456 sec. repeatedly showing the presence of identical quaternary alloy Au_{98.50}Ag_{0.80}Cu_{0.50}Zn_{0.20} as MPC-3.
Remarkably, MPC-3 being equal to MPC-2 exhibited convergence showing conclusively confirmed True Composition (TC) of alloy as Au_{98.5}Ag_{0.8}Cu_{0.5}Zn_{0.2} associated with input density D=19.0173762.
   In second step, we configured the DDS (100) to show overall final result in one go. The DDS (100) conclusively determined the True Composition (TC) of alloy Au_{98.5}Ag_{0.8} Cu_{0.5}Zn_{0.2} as converged MPC-3 after performing all the three successive steps of decoding the input density of unknown alloy within 63.6456 sec.
   Further, the DDS (100) displayed the presence of 42500 unique compositions (PICs) of alloys in Isopycnic Region (IR) at iterative step i=0.01 in the Alloy Space (AS) within the Vast Alloy Space (VAS), constituting the Quantized Field of Compositions (QFCs) in the autopoietic e-Phantom database (170). The DDS (100) also displayed percent ranges of constituent elements in these alloys at this accuracy level being Au (98.1-99.0%), Ag (0-1.9%), Cu (0-1.4%) and Zn (0-0.9%).
   The interface of DDS (100), composition spectrum and fingerprints of this alloy are illustrated in FIGS. 15A, B & C.

### Example-3

Similarly, the density of an imaginary Pt alloy containing trace metals of Au, Ag, Cu, Co, Sn, Zn and Al i.e. Pt₉₃Au₁Ag₁Cu₁Co₁Sn₁Zn₁Al₁ constituting an octonary alloy of eight metals was also calculated to be 18.67813264 using calibration module of Density Decoding System (DDS) (100).

The DDS (100) was selected for 8-Metals program at default iterative step i.e., i=1. The constituent metals Pt, of Au, Ag, Cu, Co, Sn, Zn and Al were selected along with their standard densities.
On subjecting the calculated density (D=18.67813264) as input parameter, the DDS (100) generated 27044 PICs or unique compositions in 2 out of 28 series in its Isopycnic Region (IR) of Alloy Space (AS) within the Vast Alloy Space (VAS), constituting the Quantized Field of Compositions (QFCs) in the autopoietic e-Phantom database (170). The DDS (100) also displayed percent ranges of constituent elements in these alloys at this accuracy level.
Finally, the DDS (100) conclusively determined and displayed the presence of octonary alloy Pt₉₃Au₁Ag₁ Cu₁ Co₁Sn₁Zn₁Al₁ associated with input density (D=18.67813264) in 522.675 seconds.
The density spectrum and fingerprints of this alloy are illustrated in FIGS. 13A & B.

### INDUSTRIAL APPLICABILITY

The Density Decoding System (DDS) (100) and its associated methods have broad industrial applicability across various sectors, including:
1. Materials Science and Engineering: The DDS (100) provides a powerful tool for researchers and engineers to analyze and design advanced alloys (200) with targeted densities. It enables rapid, non-destructive compositional analysis and accelerates the discovery of new alloys with unique density profiles, thereby facilitating the development of innovative materials for various applications.
2. Aerospace and Automotive Industries: The DDS (100) can be used to optimize alloy compositions for aerospace and automotive components that require specific density profiles. It enables the design (200) of lightweight, high-strength alloys, which can improve fuel efficiency, reduce emissions, and enhance the overall performance and safety of vehicles and aircraft.
3. Energy Sector: The DDS (100) can be employed in the energy sector to develop advanced alloys for power generation equipment, such as turbine blades and heat exchangers. By optimizing alloy compositions for high-temperature strength and creep resistance while maintaining desired density profiles, the DDS (100) can contribute to improving the efficiency and durability of energy systems.
4. Additive Manufacturing: The DDS (100) can be integrated with additive manufacturing processes to develop gradient alloys with spatially varying densities. By analyzing the e-Phantom database (170) and identifying composition pockets that exhibit desired density gradients, the DDS (100) can guide the design (200) and fabrication of functionally graded materials for various applications, such as aerospace components and medical implants.
5. Materials Informatics and Machine Learning: The DDS (100) generates a vast amount of data on alloy compositions, densities, and performance, which can be leveraged for materials informatics and machine learning applications. The data generated by the DDS (100) can be used to train predictive models, identify composition-density relationships, and guide the design of new alloys (200) with targeted densities, thereby accelerating materials discovery and optimization.
6. Quality Control and Failure Analysis: The DDS (100) can be employed in quality control and failure analysis processes to determine the elemental compositions of alloy components quickly and non-destructively. By comparing the determined compositions and densities with the specified standards, manufacturers can ensure that the alloys meet the required specifications and identify potential sources of failure, thereby improving product quality, reliability, and safety. It can also be used for ‘hallmarking’ of precious metals like platinum, gold, silver etc.
   The industrial applicability of the DDS (100) extends beyond these specific examples, as the system's adaptability and versatility make it valuable for any industry that relies on advanced alloys and materials. By providing a rapid, non-destructive, green, ergonomic, and cost-effective means of compositional analysis and alloy design (200) based on density, the DDS (100) has the potential to revolutionize materials development and optimization across various sectors, driving innovation and sustainability in the process. As industries continue to demand materials with tailored densities and performance, the DDS (100) is poised to play a crucial role in meeting these challenges and shaping the future of materials science and engineering.

1. A Density Decoding System (DDS) for determining elemental percent compositions of multi-component alloys from their densities, comprising:
(a) an input interface configured to receive an input density of an unknown alloy from a densitometer or any other source, and calibration parameters including a Metals program specifying number of constituent elements, the constituent elements along with their standard densities, and an iterative step (default i=1);
(b) a computing unit operatively coupled with the input interface and configured to execute:
a mathematical module (Maths-mill) for computing Probable Iso-density Compositions (PICs) for the input density of unknown alloy using modified Archimedes density equations, wherein the PICs are computed in a plurality of series based on combinatorial subset ⁿC₂ of the constituent elements, where n stands for number of constituent elements, and wherein each PIC series is computed by iteratively feeding quantized Successively increasing Predefined Imaginary Numerical values (SPIN-values) for percent mass fractions of the 3rd, 4th or additional constituent elements into the modified Archimedes density equations;
a compiler module (180) for compiling the computed PICs into respective PIC series to constitute a real-time database (i-Database) and mapping the PIC series in the Quantized Field of Compositions (QFCs) in an autopoietic e-Phantom database to visualize them as Isopycnic Regions (IRs) in an Alloy Space (AS) of a Vast Alloy Space (VAS), wherein the e-Phantom database is generated instantly upon selecting the constituent elements and iterative step, and comprises composition pockets each having an associated effective density that encodes a True Composition (TC) of the alloy constituting a Density Genome (DG) for the pocket;
an analyzer module for identifying Concordant Compositions (CCs) among the plurality of PIC series in the Isopycnic Regions (IRs) as Most Probable Composition (MPC) of unknown alloy, mapping density of the MPC to the effective densities in the QFCs pockets of the autopoietic e-Phantom database, and iteratively refining the mapped composition until convergence to determine the True Composition of unknown alloy; and for mining the e-Phantom database to identify unexplored composition pockets as potential new alloys;
(c) an output interface Gas operatively coupled with the computing unit for displaying the determined True Composition (TC) as characterisation of the unknown alloy, alloy designing data, and one or more visualizations of the PICs, CCs, density fingerprints, quantized e-Phantom database mappings, alloy composition maps highlighting unexplored pockets, spectral fingerprints, wave interference patterns, 1D/2D/3D projections, and combinations thereof;
wherein, the system (DDS) is configured to execute input interface, the mathematical module (Maths-mill), compiler module, analyzer module, and output interface without prior knowledge of the constituent elements of the unknown alloy for the n-elements program.

2. The system (DDS) according to claim 1, wherein the computing unit comprises a memory unit and a processing unit having a central processing unit (CPU) and a graphics processing unit (GPU) configured to execute the PIC computations and database mapping in parallel.

3. The system (DDS) according to claim 1, wherein the input interface, computing unit, and output interface are integrated into a stand-alone computing device.

4. The system (DDS) according to claim 1, wherein the input interface and output interface are operatively coupled with the computing unit over a computer network to form a client machine and remote/server/cloud architecture.

5. The system (DDS) according to claim 1, wherein the modified Archimedes density equations have a generalized form:
$$\begin{matrix}{m}_{1} = \frac{[ [ ( 100 - {\sum }_{i = 3}^{n} {m}_{i} ) * ( \frac{1}{{d}_{2}} ) ] - [ ( \frac{100}{D} ) - {\sum }_{i = 3}^{n} ( \frac{{m}_{i}}{{d}_{i}} ) ] ]}{( \frac{1}{{d}_{2}} - \frac{1}{{d}_{1}} )} \\ {m}_{2} = 100 - [ {\sum }_{i = 3}^{n} {m}_{i} ]\end{matrix}$$
where,
D=density of the alloy in g/cm³,
m₁, m₂, m₃ . . . mₙ=mass of constituent elements,
d₁, d₂, d₃ . . . dₙ=standard densities of constituent elements in g/cm³ (arranged in order d₁>d₂>d₃> . . . dₙ), and
n=number of constituent elements.

6. The system (DDS) according to claim 1, wherein the autopoietic e-Phantom database containing the Quantized Field of Compositions (QFCs) in the Vast Alloy Space (VAS) is generated instantly upon selecting the constituent elements and comprises:
(a) a percent grid (p-Grid) defining discrete composition pockets at a resolution determined by the iterative step, wherein each pocket represents all alloy compositions within a quantized range; and
(b) a density grid (d-Grid) specifying effective densities corresponding to each composition pocket, wherein each effective density encodes the True Composition (TC) of the alloy for its pocket and is designated as a Density Genome (DG).

7. The system (DDS) according to claim 1, wherein mapping the Most Probable Composition (MPC) to an effective density of a pocket comprises matching the density of the MPC to the effective density constituting the Density Genome (DG) in the Quantized Field of Compositions (QFCs) in the Vast Alloy Space (VAS) of the autopoietic e-Phantom database at the resolution of the selected iterative step.

8. The system (DDS) according to claim 7, wherein the iterative refinement by the analyzer module comprises:
(a) if the Most Probable Composition (MPC) density does not match the Density Genome (DG) of a pocket at the selected iterative step, then reducing the iterative step, recomputing the PICs, repeating the identification and mapping of CCs; and
(b) if the Most Probable Composition (MPC) density matches a Density Genome (DG) of a pocket at the selected iterative step, then designating the MPC as the True Composition (TC) of the alloy and terminating the process.

9. The system (DDS) according to claim 1, wherein the computing unit (165) is further configured to:
(a) demonstrate a hierarchical relationship among its various metals programs, with each higher metals program encompassing and substituting the functions of the lower metals programs;
(b) initiate each program with the assumption that the input density (may be associated with any conceivable combination of preselected elements and conduct a comprehensive procedural analysis for each input density, aiming to determine the true outcome;
(c) automatically zero out any pre-selected elements not present among the constituent elements associated with the input density from the CCs; and
(d) determine the constitution of all alloys, including binaries having singular compositions without series and individual pre-selected elements having no composition.

10. The system (DDS) according to claim 1, further comprising a calibration module configured to:
(a) allow a user to constitute imaginary alloys with a desired number of constituent elements;
(b) calculate Archimedean densities of the imaginary alloys using the equation:
$$D = \frac{M}{V} = \frac{[ {m}_{1} + {m}_{2} + {m}_{3} + … + {m}_{n} ]}{[ ( \frac{{m}_{1}}{{d}_{1}} ) + ( \frac{{m}_{2}}{{d}_{2}} ) + ( \frac{{m}_{3}}{{d}_{3}} ) + … + ( \frac{{m}_{n}}{{d}_{n}} ) ]}$$
where,
D=density of the alloy in g/cm³,
M=mass of the alloy (100 units),
m₁, m₂, m₃ . . . mₙ=mass of constituent elements,
d₁, d₂, d₃ . . . dₙ=standard densities of respective constituent elements in g/cm³, and
n=number of constituent elements;
(c) subject the calculated densities of the imaginary alloys as input parameters to the system (DDS); and
(d) verify the accuracy of the system (DDS) by comparing the elemental compositions determined by the system with the known compositions of the imaginary alloys.

11. A method for determining elemental percent compositions of multi-component alloys from their densities, comprising:
(a) receiving an input density of unknown alloy from a densitometer or any other source, and calibration parameters including a Metals program specifying number of constituent elements, the constituent elements along with their standard densities, and an iterative step (default i=1);
(b) computing Probable Iso-density Compositions (PICs) of the alloy for the input density using modified Archimedes density equations, wherein the PICs are computed in a plurality of series based on combinatorial subset ⁿC₂ of the constituent elements, where n is the number of constituent elements, and wherein each PIC series is computed by iteratively feeding quantized Successively increasing Predefined Imaginary Numerical values (SPIN-values) for percent mass fractions of the 3rd, 4th or additional constituent elements into the modified Archimedes density equations;
(c) compiling the computed PICs into respective PIC series to constitute a real-time database (i-Database) and mapping the PIC series in the Quantized Field of Compositions (QFCs) in an autopoietic e-Phantom database to visualize them as Isopycnic Regions (IRs) in an Alloy Space (AS) of a Vast Alloy Space (VAS), wherein the e-Phantom database is generated instantly upon selecting the constituent elements and iterative step, and comprises composition pockets each having an associated effective density that encodes a True Composition (TC) of the alloy constituting a Density Genome (DG) for the pocket;
(d) identifying Concordant Compositions (CCs) among the plurality of PIC series in the Isopycnic Regions (IRs), wherein the CCs replicate across the PIC series and represent a Most Probable Composition (MPC) of the alloy;
(e) mapping the MPC to an effective density in the QFCs pockets of the autopoietic e-Phantom database, wherein the effective density encodes a True Composition (TC) of the alloy for its corresponding composition pocket in the database;
(f) iteratively refining the MPC by mapping its density to the effective density at successively higher resolution levels of the e-Phantom database until convergence to determine the True Composition (TC) of the alloy;
(g) mining the e-Phantom database to identify unexplored composition pockets representing potential new alloys; and
(h) outputting the determined True Composition (TC) as characterisation of the unknown alloy, alloy designing data, and one or more visualizations of the PICs, CCs, density fingerprints, quantized e-Phantom database mappings, alloy composition maps highlighting unexplored pockets, spectral fingerprints, wave interference patterns, 1D/2D/3D projections, and combinations thereof;
wherein, steps (a) through (h) are performed without prior knowledge of the constituent elements of the unknown alloy for the n-elements program.

12. The method according to claim 11, wherein the autopoietic e-Phantom database of Quantized Field of Compositions (QFC) in Vast Alloy Space (VAS) is generated instantly upon selecting the constituent elements and comprises:
(a) a percent grid (p-Grid) defining discrete composition pockets at a resolution determined by the iterative step, wherein each pocket represents all alloy compositions within a quantized range; and
(b) a density grid (d-Grid) specifying effective densities corresponding to each composition pocket, wherein each effective density encodes the True Composition (TC) of alloy of its pocket and is designated as a density genome (DG).

13. The method according to claim 11, wherein mapping the Most Probable Composition (MPC) to an effective density of pocket comprises matching the density of the MPC to the effective density constituting the Density Genome (DG) in the Quantized Field of Compositions (QFCs) in Vast Alloy Space (VAS) of autopoietic e-Phantom database at the resolution of the selected iterative step.

14. The method according to claim 13, wherein the iterative refinement comprises:
(a) if the Most Probable Composition (MPC) density does not match Density Genome (DG) of pocket at the selected iterative step, then reducing the iterative step, recomputing the PICS, and repeating steps (c) through (e); and
(b) if the Most Probable Composition (MPC) density matches a Density Genome (DG) of pocket at the selected iterative step, then designating the Most Probable Composition (MPC) as the True Composition (TC) of input density of unknown alloy and terminating the method.

15. The method according to claim 11, further comprising:
(a) exploring the unexplored composition pockets to identify potential new alloys; and
(b) synthesizing and testing the potential new alloys to determine their properties and performance.

16. The method according to claim 11, wherein analyzing the autopoietic e-Phantom database comprises:
(a) training a machine learning model on the composition-density relationships encoded in the database;
(b) using the trained model to predict the density values for unexplored composition pockets; and
(c) identifying composition pockets that exhibit targeted density values or ranges based on the predictions.

17. A non-transitory computer readable medium with computer executable instructions stored thereon for determining elemental percent compositions of multi-component alloys from their densities, wherein the instructions, when executed by a processor, cause the processor to perform the following method:
(a) receiving an input density of unknown alloy from a densitometer or any other source, and calibration parameters including a Metals program specifying number of constituent elements (150), the constituent elements along with their standard densities, and an iterative step (default i=1);
(b) computing Probable Iso-density Compositions (PICS) of the alloy for the input density using modified Archimedes density equations, wherein the PICs are computed in a plurality of series based on combinatorial subset ⁿC₂ of the constituent elements, where n is the number of constituent elements, and wherein each PIC series is computed by iteratively feeding quantized Successively increasing Predefined Imaginary Numerical values (SPIN-values) for percent mass fractions of the 3rd, 4th or additional constituent elements in to the modified Archimedes density equations;
(c) compiling the computer PICs into respective PIC series to constitute a real-time database (i-Database) and mapping the PIC series in the Quantized Field of Compositions (OFCs) in an autopoietic e-Phantom database to visualize them as Isopycnic Regions (IRs) in an Alloy Space (AS) of a Vast Alloy Space (VAS), wherein the e-Phantom database is generated instantly upon selecting the constituent elements and iterative step, and comprises composition pockets each having an associated effective density that encodes a True Composition (TC) of the alloy constituting a Density Genome (DG) for the pocket;
(d) identifying Concordant Compositions (CCs) among the plurality of PIC series in the Isopycnic Regions (IRs), wherein the CCs replicate across the PIC series and represent a Most Probable Composition (MPC) of the alloy;
(e) mapping the MPC to an effective density in the QFCs pockets of the autopoietic e-Phantom database, wherein the effective density encodes a True Composition (TC) of the alloy for its corresponding composition pocket in the database,
(f) iteratively refining the MPC by mapping its density to the effective density at successively higher resolution levels of the e-Phantom database until convergence to determine the True Composition (TC) of the alloy;
(g) mining the e-Phantom database to identify unexplored composition pockets representing potential new alloys; and
(h) outputting the determined True Composition (TC) as characterizations of the unknown alloy, alloy designing data, and one or more visualizations of the PICs, CCs, density fingerprints, quantized e-Phantom database mappings, alloy composition maps highlighting unexplored pockets, spectral fingerprints, wave interference patterns, 1D/2D/3D projections, and combinations thereof;
wherein, steps (a) through (b) are preformed without prior knowledge of the constituent elements of the unknown alloy for the n-elements program.

12. The method according to claim 11, wherein the autopoietic e-Phantom database of Quantized Field of Compositions (QFC) in Vast Alloy Space (VAS) is generated instantly upon selecting the constituent elements and comprises:
(a) a percent grid (p-Grid) defining discrete composition pockets at a resolution determined by the iterative step, wherein each pocket represents all alloy compositions within a quantized range, and
(b) a density grid (d-Grid) specifying effective densities corresponding to each composition pocket, wherein each effective density encodes the True Composition (TC) of alloy of its pocket and is designated as a density genome (DG).

18. The system (DDS) (100) according to claim 1, the further comprising:
(a) selecting a target density;
(b) providing a target value or range for the density;
(c) analyzing the autopoietic e-Phantom database to identify composition pockets that exhibit the target density value or range;
(d) extracting the elemental percent ranges associated with the identified composition pockets; and
(e) designing an alloy by selecting elemental percentages within the extracted ranges.

19. The system (DDS) (100) according to claim 18, further comprising:
(a) synthesizing the designed alloy;
(b) measuring the actual density value of the synthesized alloy;
(c) comparing the actual density value with the target value or range; and
(d) iteratively refining the alloy design based on the comparison.

20. The system (DDS) (100) according to claim 1, further comprising:
(a) analyzing the autopoietic e-Phantom database to identify composition pockets that exhibit uncommon density values;
(b) extracting the elemental percent ranges associated with the identified composition pockets;
(c) designing a set of candidate alloys by selecting elemental percentages within the extracted ranges; and
(d) synthesizing and testing the candidate alloys to validate their densities.

21. The system (DDS) (100) according to claim 20, further comprising:
(a) characterizing the microstructure and phase composition of the validated alloys;
(b) correlating the microstructure and phase composition with the measured densities; and
(c) refining the alloy design based on the structure-density correlations.

22. The system (DDS) according to claim 1, her comprising:
(a) providing a set of application-specific density requirements;
(b) analyzing the autopoietic e-Phantom database to identify composition pockets that satisfy the application-specific density requirements;
(c) extracting the elemental percent ranges associated with the identified composition pockets;
(d) designing a set of candidate alloys by selecting elemental percentages within the extracted ranges;
(e) ranking the candidate alloys based on their predicted density performance, cost, and manufacturability; and
(f) selecting the top-ranked alloys for further development and testing.

23. A computer-implemented method for determining elemental percent compositions of multi-component alloys from their densities using the Density Decoding System (DDS), the method comprising:
(a) receiving, via an input interface, an input density of an alloy and calibration parameters including a Metals program; specifying number of constituent elements, the constituent elements along with their standard densities, and an iterative step;
(b) computing, using a processor executing a mathematical module (Maths-mill), Probable Iso-density Compositions (PICs) of the alloy for the input density; using modified Archimedes density equations, wherein the PICs are computed in a plurality of series based on combinatorial subset ⁿC₂ of the constituent elements, where n is the number of constituent elements, and wherein each PIC series is computed by iteratively feeding quantized Successively increasing Predefined Imaginary Numerical values (SPIN-values) for percent mass fractions of additional constituent elements into the modified Archimedes density equations;
(c) compiling, using the processor executing a compiler module, the computed PICs into respective PIC series to constitute a real-time database and mapping the PIC series in the Quantized Field of Compositions (QFCs) in an autopoietic e-Phantom database to visualize them as Isopycnic Regions (IRs) in an Alloy Space (AS) of a Vast Alloy Space (VAS);
(d) identifying, using the processor executing an analyzer module, Concordant Compositions (CCs) among the plurality of PIC series, mapping a density of the CCs to the effective densities in the QFCs pockets of the autopoietic e-Phantom database, and iteratively refining the mapped composition until convergence to determine the True Composition (TC) of the alloy;
(e) mining, using the processor executing the analyzer module, the e-Phantom database to identify unexplored composition pockets as potential new alloys; and
(f) outputting, via an output interface, the determined True Composition (TC) of unknown alloy as characterisation, alloy designing data, and one or more visualizations.

24. The computer-implemented method of claim 23, wherein the modified Archimedes density equations have a generalized form:
$$\begin{matrix}{m}_{1} = \frac{[ [ ( 100 - {\sum }_{i = 3}^{n} {m}_{i} ) * ( \frac{1}{{d}_{2}} ) ] - [ ( \frac{100}{D} ) - {\sum }_{i = 3}^{n} ( \frac{{m}_{i}}{{d}_{i}} ) ] ]}{( \frac{1}{{d}_{2}} - \frac{1}{{d}_{1}} )} \\ {m}_{2} = 100 - [ {\sum }_{i = 3}^{n} {m}_{i} ]\end{matrix}$$
where,
D=density of the alloy in g/cm³,
m₁, m₂, m₃ . . . mₙ=mass of constituent elements,
d₁, d₂, d₃ . . . dₙ=standard densities of constituent elements in g/cm³ (arranged in order d₁>d₂>d₃> . . . dₙ), and
n=number of constituent elements.

25. The computer-implemented method of claim 23, wherein mapping the Most Probable Composition (MPC) to an effective density of a pocket comprises matching the density of the MPC to the effective density constituting the Density Genome (DG) in the Quantized Field of Compositions (QFCs) in the Vast Alloy Space (VAS) of the autopoietic e-Phantom database at the resolution of the selected iterative step.

26. The computer-implemented method of claim 23, further comprising:
(a) selecting a target density;
(b) providing a target value or range for the density;
(c) analyzing the autopoietic e-Phantom database to identify composition pockets that exhibit the target density value or range;
(d) extracting the elemental percent ranges associated with the identified composition pockets; and
(e) designing an alloy by selecting elemental percentages within the extracted ranges.

27. A computer program product for determining elemental percent compositions of multi-component alloys from their densities using the Density Decoding System (DDS), the computer program product comprising a non-transitory computer readable medium having program instructions embodied therewith, the program instructions executable by a processor to cause the processor to perform a method comprising:
(a) receiving an input density of an unknown alloy; and calibration parameters including a Metals program specifying number of constituent elements, the constituent elements along with their standard densities, and an iterative step;
(b) computing Probable Iso-density Compositions (PICs) of the alloy for the input density using modified Archimedes density equations, wherein the PICs are computed in a plurality of series based on combinatorial subset ⁿC₂ of the constituent elements, where n is the number of constituent elements, and wherein each PIC series is computed by iteratively feeding quantized Successively increasing Predefined Imaginary Numerical values (SPIN-values) for percent mass fractions of additional constituent elements into the modified Archimedes density equations;
(c) compiling the computed PICs into respective PIC series to constitute a real-time database and mapping the PIC series in the Quantized Field of Compositions (QFCs) in an autopoietic e-Phantom database to visualize them as Isopycnic Regions (IRs) in an Alloy Space (AS) of a Vast Alloy Space (VAS);
(d) identifying Concordant Compositions (CCs) among the plurality of PIC series, mapping density of the CCs to the effective densities in the QFCs pockets of the autopoietic e-Phantom database, and iteratively refining the mapped composition until convergence to determine the True Composition (TC) of the alloy;
(e) mining the e-Phantom database to identify unexplored composition pockets as potential new alloys; and
(f) outputting the determined True Composition (TC) as characterisation of the unknown alloy, alloy designing data, and one or more visualizations.

28. The computer program product of claim 27, wherein the method further comprises:
(a) selecting a target density;
(b) providing a target value or range for the density;
(c) analyzing the autopoietic e-Phantom database to identify composition pockets that exhibit the target density value or range;
(d) extracting the elemental percent ranges associated with the identified composition pockets; and
(e) designing an alloy by selecting elemental percentages within the extracted ranges.

29. The computer program product of claim 27, wherein the method further comprises:
(a) analyzing the autopoietic e-Phantom database to identify composition pockets that exhibit uncommon density values;
(b) extracting the elemental percent ranges associated with the identified composition pockets;
(c) designing a set of candidate alloys by selecting elemental percentages within the extracted ranges; and
(d) synthesizing and testing the candidate alloys to validate their densities.

30. A computer system for determining elemental percent compositions of multi-component alloys from their densities using the Density Decoding System (DDS), the system comprising:
(a) a processor;
(b) an input interface coupled to the processor and configured to receive an input density of unknown alloy and calibration parameters including a Metals program specifying number of constituent elements, the constituent elements along with their standard densities, and an iterative step;
(c) an output interface coupled to the processor; and
(d) a memory coupled to the processor, wherein the memory stores instructions which, when executed by the processor, cause the processor to:
compute Probable Iso-density Compositions (PICs) of the alloy for the input density (20) using modified Archimedes density equations, wherein the PICs are computed in a plurality of series based on combinatorial subset ⁿC₂ of the constituent elements, where n is the number of constituent elements, and wherein each PIC series is computed by iteratively feeding quantized Successively increasing Predefined Imaginary Numerical values (SPIN-values) for percent mass fractions of additional constituent elements into the modified Archimedes density equations;
compile the computed PICs into respective PIC series to constitute a real-time database and map the PIC series in the Quantized Field of Compositions (QFCs) in an autopoietic e-Phantom database to visualize them as Isopycnic Regions (IRs) in an Alloy Space (AS) of Vast Alloy Space (VAS);
identify Concordant Compositions (CCs) among the plurality of PIC series, map a density of the CCs to the effective densities in the QFCs pockets of the autopoietic e-Phantom database, and iteratively refine the mapped composition until convergence to determine the True Composition (TC) of the alloy;
mine the e-Phantom database to identify unexplored composition pockets as potential new alloys; and
output, via the output interface, the determined True Composition (TC) of unknown alloy as characterisation, alloy designing data, and one or more visualizations.

31. The computer system of claim 30, wherein the memory stores further instructions which, when executed by the processor, cause the processor to:
(a) select a target density;
(b) provide a target value or range for the density;
(c) analyze the autopoietic e-Phantom database to identify composition pockets that exhibit the target density value or range;
(d) extract the elemental percent ranges associated with the identified composition pockets; and
(e) design an alloy by selecting elemental percentages within the extracted ranges.

32. The computer system of claim 30, wherein the memory stores further instructions which, when executed by the processor, cause the processor to:
(a) analyze the autopoietic e-Phantom database to identify composition pockets that exhibit uncommon density values;
(b) extract the elemental percent ranges associated with the identified composition pockets;
(c) design a set of candidate alloys by selecting elemental percentages within the extracted ranges; and
(d) synthesize and test the candidate alloys to validate their densities.

33. A method for developing gradient alloys with spatially varying densities using the Density Decoding System (DDS) according to claim 1, the method comprising:
(a) selecting a set of target densities that vary spatially according to application requirements;
(b) analyzing the autopoietic e-Phantom database to identify composition pockets that exhibit the desired spatial variation of densities;
(c) extracting the elemental percent ranges associated with the identified composition pockets;
(d) designing a gradient alloy by selecting elemental percentages within the extracted ranges and specifying their spatial distribution; and
(e) synthesizing the gradient alloy using additive manufacturing or other suitable techniques.

34-40. (canceled)
